SEQUENCE LISTING (1 sequence)

SEQ ID NO: 1 - protein, 42 aa, Artificial
  Synthetic
MKMMMKKCKMCKCMKMCCCMCCMMCCKKKKKKMKKKKKKKMK

(19) United States
(12) Patent Application Publication
(10) Pub. No.: US 2022/0036973 A1
(43) Pub. Date: Feb. 3, 2022

(54) MACHINE LEARNING FOR PROTEIN IDENTIFICATION

(71) Applicant: TECHNION RESEARCH & DEVELOPMENT FOUNDATION LIMITED, Haifa (IL)

(72) Inventors: Amit MELLER, Haifa (IL); Shilo OHAYON, Lod (IL); Arik GIRSAULT, Haifa (IL)

(21) Appl. No.: 17/288,539

(22) PCT Filed: Oct. 24, 2019
(86) PCT No.: PCT/IL2019/051149

§ 371 Date: Apr. 25, 2021

Related U.S. Application Data
(60) Provisional application No. 62/750,357, filed on Oct. 25, 2018.
(60) Provisional application No. 62/753,140, filed on Oct. 31, 2018.

(51) Int. Cl. G16B 40/00; G16B 30/00

(52) U.S. Cl. CPC G16B 40/00; G16B 30/00

(57) ABSTRACT

Methods for identifying a peptide by analyzing a linear readout representative of at least a portion of at least two amino acids along the peptide using a machine learning model, wherein the machine learning model is trained on linear readouts representative of a set of peptides of known sequence are provided. Methods of training a machine learning model on linear readouts representative of a set of known peptides, and systems for performing the methods of the invention are also provided.

### CROSS REFERENCE TO RELATED APPLICATIONS

This application claims the benefit of priority of U.S. Provisional Patent Application Nos. 62/750,357, filed Oct. 25, 2018, and 62/753,140, filed Oct. 31, 2018, the contents of which are all incorporated herein by reference in their entirety.

### FIELD OF INVENTION

The present invention is in the field of machine learning and nanopore-based protein sequencing.

### BACKGROUND OF THE INVENTION

Modern DNA sequencing techniques have revolutionized genomics, but extending these methods to routine proteome analysis, and specifically to single-cell proteomics, remains a global unmet challenge. This is attributed to the fundamental complexity of the proteome: protein expression level spans several orders of magnitude, from a single copy to tens of thousands of copies per cell; and the total number of proteins in each cell is staggering. Given the lack of in-vitro protein amplification assays the ability to accurately quantify both abundant and rare proteins hinges on the development of single-protein identification methods that also feature extraordinary-high sensing throughput. To date, however, protein sequencing techniques, such as mass-spectrometry, have not reached single-molecule resolution, and rely on bulk averaging from hundreds of cells or more. Affinity-based method can reach single protein sensitivity, but depend on limited repertoires of antibodies, thus severely hindering their applicability for proteome-wide analyses. Consequently, in the past few years single-molecule approaches for proteome analysis based on Edman degradation or FRET have been proposed. To date, however, profiling of the entire proteome of individual cells remains the ultimate challenge in proteomics.

Nanopores are single-molecule biosensors adapted for DNA sequencing, as well as other biosensing applications. Recent nanopore studies extended nucleic-acid detection to proteins, demonstrating that ion current traces contain information about protein size, charge and structure. However, to date, the challenge of deconvolving the electrical ion-current trace to determine the protein's amino-acid sequence from the time-dependent electrical signal has remained elusive. In an analogy to the field of transcriptomics, in many practical cases it is sufficient to identify and quantify each protein among the repertoire of known proteins, instead of re-sequencing it. It has been shown that theoretically most, but not all, proteins in the human proteome database can be uniquely identified by the order of appearance of just two amino-acids, lysine and cysteine (K and C, respectively). However, taking into account common experimental errors, for example due to false calling of an amino-acid, or an unlabeled amino-acid, sharply reduces the identification accuracy. A protein identification method that correctly identifies all proteins and remains robust against the expected experimental errors is greatly needed.

### SUMMARY OF THE INVENTION

The present invention provides methods and systems for identifying a peptide by analyzing a linear readout representative of at least a portion of at least two amino acids along the peptide using a machine learning model, wherein the machine learning model is trained on linear readouts representative of a set of peptides of known sequence. Methods of training a machine learning model on linear readouts representative of a set of known peptides are also provided.

According to a first aspect, there is provided a method of identifying a peptide, comprising:
a. receiving a linear readout representative of at least a portion of a first amino acid and at least a portion of a second amino acid along the peptide; and
b. analyzing the linear readout with a machine learning model, wherein the machine learning model predicts the identity of the peptide;
thereby identifying a peptide.

According to another aspect, there is provided a method comprising:

at a training stage, training a machine learning model on a training set comprising:
(i) a plurality of linear readouts, each representing at least a portion of a first amino acid and at least a portion of a second amino acid along a peptide, and
(ii) labels identifying the peptide associated with each of the linear readouts; and
at an inference stage, applying the trained machine learning model to a target linear readout representing at least a portion of the first amino acid and at least a portion of the second amino acid along a target peptide, to identify the target peptide.

According to another aspect, there is provided a system comprising:

at least one hardware processor; and

a non-transitory computer-readable storage medium having stored thereon program instructions, the program instructions executable by the at least one hardware processor to:

train a machine learning model based, at least in part, on a training set comprising:
(i) a plurality of linear readouts, each representing at least a portion of a first amino acid and at least a portion of a second amino acid along a peptide, and
(ii) labels identifying the peptide associated with each of the linear readouts; and
apply the machine learning model to a target linear readout representing at least a portion of the first amino acid and at least a portion of the second amino acid along a target peptide, to identify the target peptide.

According to some embodiments, the portion is at least 60%. According to some embodiments, the portion of the first amino acid is at least 60%. According to some embodiments, the portion of the second amino acid is at least 60%. According to some embodiments, the portion is at least 80%. According to some embodiments, the portion of the first amino acid is at least 80%. According to some embodiments, the portion of the second amino acid is at least 90%.

According to some embodiments, the machine learning model is trained on linear readouts of a set of peptides, wherein each linear readout represents at least a portion of the first amino acid and at least a portion of the second amino acid along a peptide from the set of peptides.

According to some embodiments, the method of the invention further comprises labeling at least a portion of the first amino acid with a first label and at least a portion of the second amino acid with a second label along the peptide.

According to some embodiments, the method of the invention further comprises detecting the first and second label linearly along the peptide to produce the readout.

According to some embodiments, the detecting comprises passing the labeled peptide though a nanopore, wherein the first and second labels are uniquely detectable as each label passes through the nanopore.

According to some embodiments, the label comprises a fluorophore and an optical sensor at the nanopore is configured to detect fluorescence at the nanopore.

According to some embodiments, the label is a bulky group and an electrical sensor at the nanopore is configured to detect electrical current and/or voltage at the nanopore.

According to some embodiments, the nanopore contains a plasmonic nanostructure, wherein the plasmonic nanostructure is configures to localize electromagnetic excitation below a wavelength of light. According to some embodiments, the plasmonic nanostructure is configures to amplify localized fluorescence emission at the nanopore at a plurality of wavelengths.

According to some embodiments, the nanopore has a resolution of at least 100 nm.

According to some embodiments, the linear readout is a linear temporal trace of the peptide as it passes through a nanopore.

According to some embodiments, the peptide is an undigested or unfragmented protein.

According to some embodiments, the linear readout is further representative of a portion of at least a third amino acid along the peptide.

According to some embodiments, the first, second and third amino acids are lysine, cysteine and methionine.

According to some embodiments, the set of peptides is a set of peptides selected from:
a. a set of peptides with known sequences;
b. a set of peptides expected to be in a sample and wherein the peptide is from the sample;
c. proteins found in plasma and wherein the peptide is a peptide found in plasma; and
d. proteins found in a proteome and wherein the peptide is from the proteome.

According to some embodiments, the linear readouts of a set of peptides comprise at least 50 linear readouts representative of each peptide from the set.

According to some embodiments, the linear readouts of a set of peptides are simulated linear readouts based on a known sequence for each peptide wherein at least a portion of the first amino acid and a portion of the second amino acid are represented in the simulated readout.

According to some embodiments, the training set comprises linear readouts of a set of peptides expected to be in a sample and the target peptide is from the sample.

According to some embodiments, the training set comprises linear readouts of all proteins found in plasma, or all proteins found in a proteome.

According to some embodiments, the training set comprises linear readouts for at least 15 peptides and at least 50 readouts for each peptide.

According to some embodiments, the linear readouts are simulated linear readouts generated by selecting a known sequence of a peptide and generating a linear representation of at least a portion of the first amino acids and at least a portion of the second amino acids along the peptide.

According to some embodiments, the liner readouts further represent at least a portion of a third amino acid along the peptide.

According to some embodiments, the linear readouts comprise a linear temporal trace of a labeled peptide as it passes through a nanopore, wherein the peptide is labeled at least at a portion of the first amino acid and at least at a portion of the second amino acid along the peptide.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-C: An overview of the Nanopore, tri-color protein identification method. (1A) A tentative sample process flow. The protein sample is first denatured using SDS and cysteines (C), lysines (K) and methionines (M) are labeled with three spectrally-resolvable fluorophores (blue-B; red-R; green-G). The labeled, SDS-denatured proteins are then threaded through a nanopore and excited by a laser light focused by a plasmonic architecture. The plasmonic field ensures local excitation of small portions of the denatured proteins. Finally, the photon emissions from each protein are measured in three channels, one for each fluorophore, to create a tri-color optical trace per translocation. (1B) A pre-trained convolutional neural network (CNN) classifier subsequently examines and classifies each trace, extracting its relevant features using a convolutional, an activation, a pooling and a fully connected layer, to identify the protein. (1C) A theoretical evaluation of whole proteome fingerprinting based on complete labeling of C and K or C, K and M amino acids. Using only counts of the number of labeled Cs and Ks yields unique identifications (ID) of 51% of all proteins. Counting only the number of labeled Cs, Ks and Ms yields unique ID of 72% of all proteins. The remaining 28% of proteins were not uniquely identified and were either identified as one out of two (green slice, labeled “2”) or more proteins as indicated by color and label. Considering also the order of the three labeled amino acids increases the unique ID fraction to 99%.

FIGS. 2A-E: Simulation of the fluorescence signals generated during the translocation of the SDS-denatured PH and SECT domain-containing (PSD) protein. (2A) The nanopore diameter and height were set to 3 and 5 nm, respectively, and the plasmonic architecture deposited on its ‘top-side’ produced a confined excitation profile (14-20 nm axial full-width half maximum) whose color map displayed on the left indicates the excitation near field enhancement at a wavelength of 640 nm (modeled using FDTD, see FIG. 2E). Two snapshots of the translocation process are shown and denoted by the timepoints t₀ and t₁ at which they were respectively taken. Energy transfer, photo-bleaching, incomplete labeling and non-specific labeling are indicated by dotted lines, solid grey, purple and green arrows, respectively. (2B) Zoomed in region of the polypeptide in which Forster resonance energy transfer (FRET) is shown in greater details. In this configuration, energy was transferred from lysine fluorophores to cysteine and methionine emitters, and from cysteine to methionine fluorophores. (2C) The fluorescence emission rate of each labeled amino-acid was modeled as either a two-state or three-state system (see online methods for further details and in which k_{F+} and k_{F-} refer to k_{FRET,+} and k_{FRET,-}, respectively). kₑₓₑ denotes the absorption rate, k_{isc} the inter-system crossing rate and k_{T1} the triplet state relaxation rate. Fluorophores are depicted in a color which denote the excitation wavelength with which they are excited or the channel to which they belong. (2D) Schematics of the nanopore chip and optical system, which includes a high NA water immersion objective lens, three excitation laser lines (640-red, 561-green, 488-blue) and corresponding APDs. The nanopore chip is made of four consecutive layers: silicon, silicon nitride in which the nanopore is drilled, titanium oxide and gold. (2E) (Upper) Near Field Enhancement along the z-profile (direction of biopolymer translocation) calculated using FDTD simulations. The near field enhancement can be approximated by a Gaussian function whose full-width-half-maximum (FWHM) is 14 nm. For the protein fingerprinting simulations, a minimal FWHM of 20 nm was used. (Lower) Near Field Enhancement along the x-profile of the 3 nm-wide nanopore calculated using FDTD simulations.

FIGS. 3A-B: Measurements of SDS-denatured human serum albumin translocations through solid-state nanopores. (3A) Electrical events of albumin translocating through a 4 nm-wide nanopore measured at 300 mV. (3B) Scatter plot of the fractional blockade current I_{B} versus the translocation time t, with its corresponding density map. The number of translocations events displayed amounts to 900. The inset shows the dwell-time histogram, fitted to an exponential decay with characteristic time of 94.3±7.2 μs.

FIGS. 4A-F: Simulated optical traces of epidermal growth factor (EGF) precursor protein and its receptor EGFR produced under different conditions. The C, K and M amino acids were labeled using three different fluorophores as indicated (C-green, K-blue, M-red). (4A) Optical signals simulated using a spatial resolution of 0.5 nm and a labelling efficiency of 100%. (4B) Optical signals simulated using three distinct spatial resolutions: 10, 30 and 50 nm (from left to right). At superior resolution (i.e. lower resolution) individual peaks are more apparent, however a clearly definable trace is still visible at poorer resolution. (4C) Simulated optical traces of the epidermal growth factor (EGF) precursor protein and its receptor EGFR generated using two spatial resolutions: 100 and 150 nm. The labeling efficiency was set to 100% and the average translocation velocity to 0.0035 cm/s. Even at these poor resolutions the two very similar proteins are clearly distinguishable. (4D) Bar chart of whole-proteome protein identification accuracy as a function of amino-acid dwell time and labelling efficiency. The spatial resolution was fixed to 30 nm and the dwell-time was defined as the time it took a peptide to translocate over the length of a single amino acid. The corresponding translocation velocities are 2, 0.2 and 0.035 cm/s. The APD binning was set to 1 μs. The CNN classification was still robust to low labeling efficiency and realistic spatial and temporal resolutions, expected in real experiments. (4E) Simulated optical traces of the epidermal growth factor (EGF) precursor protein in different experimental conditions. (Upper) Optical signals simulated using a spatial resolution of 0.5 nm and a labelling efficiency of 100%. (Lower) optical signals simulated using three distinct spatial resolutions: 10, 30 and 50 nm (first row), three distinct labeling efficiencies: 90%, 80% and 70% (second row), three velocity fluctuation: 20%, 30% and 40% of the mean translocation velocity v=0.035 cm/s (third row). Even at worse resolution, labeling and speeds distinct traces are clearly observed. Alterations in speed have almost no effect on the trace. (4F) Simulated optical traces of the B Double Prime 1 (BDP1) protein in different experimental conditions. (Upper) Optical signals simulated using a spatial resolution of 0.5 nm and a labelling efficiency of 100%. (Lower) optical signals simulated using three distinct spatial resolutions: 10, 30 and 50 nm (first row), three distinct labeling efficiencies: 90%, 80% and 70% (second row), three velocity fluctuation: 20%, 30% and 40% of the mean translocation velocity v=0.035 cm/s (third row). Once again, distinct traces are observable even in poor conditions.

FIG. 5: Pearson correlation among pairs of five simulated proteins photon traces. The elements of the correlation matrix, consisting of all Pearson correlation coefficients between all pairs of 50 translocation repeats, were first transformed to Fisher's z, subsequently averaged and finally transformed back into an “average” Pearson correlation coefficient. The standard deviation is given in parentheses.

FIGS. 6A-I: CNN-based classification results of whole proteome, plasma proteome, and a cytokine panel. (6A-B) The fractions of the correctly identified translocation events from whole-proteome classifications repeated five times are shown in (6A) and (6B) left panels. Each classification consisted of five separate training-and-testing of a CNN using 100 translocation events per protein (a total of ˜10⁷ events), whose resulting correct identifications were averaged. These experiments and analyses were performed under four different spatial resolutions (20, 30, 50 and 100 nm) and labelling efficiencies (60, 70, 80 and 90%). Right-hand panels show the fraction of the proteome correctly identified with probability p when considering a spatial resolution of 30 nm for different labeling efficiencies. The bin size was set to 1%. The insets display the degree of randomness in misclassification. The bin height is given by the fraction of mis-identified proteins R (i.e. proteins that had at least 10% of their events misclassified) at different rᵢ (fraction of identical mismatch) intervals: rᵢ=maxⱼ nᵢⱼ/Nᵢ for each protein i, where nᵢⱼ is the number of translocation events misidentified to protein j and Alᵢ the total number of misclassified translocation events. The bin width—rᵢ interval size—was set to 10%. The value in parentheses indicate the percentage of mis-identified proteins of a whole-proteome experiment. Other experimental conditions are provided in FIG. 6E-F. (6C) Cytokines panel identification using the same proteins as in the ELISA set “CytokineMAP A”. The heat-map represents the correct ID of each cytokine under the specified labelling efficiency and resolution. The average correct ID is provided in the right-hand column. As the labeling efficiency is increased, and as the resolution decreases (improves) the correct identification % is increased. All of the cytokines are uniquely identifiable. (6D) Bar graphs of whole-proteome probability density function of correct identification and degree of randomness in misclassification at 30 nm. (upper) The fraction of the whole proteome that was correctly identified with probability p and (lower) the degree of randomness in misclassification were determined for 30 nm and four labeling efficiencies (60, 70, and 90%; the remaining 80% as well as the CNN accuracy bar plot are shown in 6A). (6E) Bar graphs of whole-proteome degree of randomness in misclassification for different experimental conditions. For 6D-E and 6G-H: The bin size was set to 1% in all histograms. The bin height of histograms in the lower panel is given by the fraction of mis-identified proteins R (i.e. proteins that had at least 10% of their events misclassified) at different rᵢ (fraction of identical mismatch) intervals: rᵢ=ⱼ^{max} nᵢⱼ/Nᵢ for each protein i, where nᵢⱼ is the number of translocation events misidentified to protein j and Nᵢ the total number of mis-classified translocation events. High is characteristic of a low degree of randomness, and vice-versa low of a high degree of randomness. The bin width—rᵢ interval size—was set to 10%. The value in parentheses indicate the percentage of mis-identified proteins of a whole-proteome experiment. (6F) Bar charts of whole-proteome probability density function of correct identification for different experimental conditions. The fraction of the proteome that was correctly identified with probability p was determined for three spatial resolutions (20, 50 and 100 nm; 30 nm shown in article) and four labeling efficiencies (60, 70, 80 and 90%). The bin size was set to 1% in all histograms. (6G) Same as in 6D, but for plasma-proteome. (6H) Same as in 6E, but for plasma-proteome. (6I) Same as in 6F, but for plasma-proteome.

FIGS. 7A-C: Identification of proteins targeted by different commercial ELISA sets. (7A) Heatmap of whole-proteome CNN accuracy of the CytokineMAP B kit proteins for four spatial resolutions (20, 30, 50 and 100 nm) and four labeling efficiencies (60, 70, 80 and 90%). Results are similar to those reported in 6C. (7B) Heatmap of whole-proteome CNN accuracy of the MetabolicMAP kit proteins for four spatial resolutions (20, 30, 50 and 100 nm) and four labeling efficiencies (60, 70, 80 and 90%). Results are similar to those reported in 6C. (7C) Heatmap of whole-proteome CNN accuracy of the NeuroMAP A kit proteins and misclassification distribution for four spatial resolutions (20, 30, 50 and 100 nm) and four labeling efficiencies (60, 70, 80 and 90%). Results are similar to those reported in 6C.

FIG. 8: Simulated optical traces of different proteins with or without a fluorophore triplet state. The spatial resolution and labeling efficiency were fixed in all cases to 30 nm and 100%, respectively. Left column shows the simulated traces optical traces using a two-state (ground and excited) fluorophore model; right column using a three-state (ground, excited and triplet) model. Transition rates in between all states were determined according to the manufacturer (when available) and to published works.

### DETAILED DESCRIPTION OF THE INVENTION

The present invention, in some embodiments, provides methods for identifying a peptide by analyzing a linear readout representative of at least a portion of at least two amino acids along the peptide using a machine learning model, wherein the machine learning model is trained on linear readouts representative of a set of peptides. Methods of training a machine learning model on linear readouts representative of a set of known peptides, as well as systems for performing the methods of the invention are also provided.

The present invention is based on the surprising finding that by using machine learning models trained on linear representations of only a portion of a few amino acids in a peptide, peptides with imperfect labeling and/or imperfect detection conditions can be accurately identified. Identifying proteins by perfectly labeling two amino acids throughout the protein chain and then generating the exact order and position of those two amino acids is known in the art. However, in practice 100% labeling is almost never achieved and thus a degenerate readout with only some of the amino acids accounted for is what needs to be analyzed. Further, detection apparatuses are not 100% accurate either, and often have suboptimal resolution. This can lead to missing of a labeled amino acid, or discrepancies in the order/position. Generally, the variation and lack of reproducibility from one experiment to the next and one laboratory to the next, makes analyzing peptides by labeling only two amino acids not currently feasible.

However, by using a machine learning model even very degenerate readouts for peptides can be correctly identified. In the instant invention, a machine learning model is trained on numerous readouts of peptides/proteins where conditions are not ideal, but when the input peptide/protein is known. Thus, when an unknown sample is analyzed by the model, even is the sample is also poorly labeled or scanned, the machine learning model is still able to identify the peptide/protein with very high accuracy. The feasibility of this approach has been confirmed with a training set of the full human proteome, and for analysis of not only the whole human proteome, but also the plasma proteome and a panel of cytokines.

By a first aspect, there is provided a method comprising, analyzing a readout representative of at least a portion of a first amino acid along a peptide with a machine learning model, wherein the machine learning model predicts the identity of the peptide.

According to another aspect, there is provided a method comprising:
operating at least one hardware processor for:
   receiving, as input, a plurality of electronic documents, training a machine learning model based, at least in part, on a training set comprising:
   (i) labels associated with the electronic documents, and
   (ii) readouts representative of at least a portion of a first amino acid along a peptide from each of the plurality of electronic documents, and
   applying the machine learning model to classify one or more new electronic documents comprising a readout.

According to another aspect, there is provided a method comprising:

at a training stage, training a machine learning model on a training set comprising:
(i) a plurality of linear readouts, each representing at least a portion of a first amino acid along a peptide, and
(ii) labels identifying the peptide associated with each of the linear readouts; and

at an inference stage, applying the trained machine learning model to a target linear readout representing at least a portion of the first amino acid along a target peptide, to identify the target peptide

According to another aspect, there is provided a system comprising:
at least one hardware processor; and
a non-transitory computer-readable storage medium having stored thereon program instructions, the program instructions executable by the at least one hardware processor to:
receive, as input, a plurality of electronic documents,
train a machine learning model based, at least on part, on a training set comprising:
   (i) labels associated with the electronic documents, and
   (ii) readouts representative of at least a portion of a first amino acid along a peptide from each of the plurality of electronic documents, and
   apply the machine learning model to classify one or more new electronic documents comprising a readout.

According to another aspect, there is provided a system comprising:

at least one hardware processor; and

a non-transitory computer-readable storage medium having stored thereon program instructions, the program instructions executable by the at least one hardware processor to:

train a machine learning model based, at least in part, on a training set comprising:
(i) a plurality of linear readouts, each representing at least a portion of a first amino acid along a peptide, and
(ii) labels identifying the peptide associated with each of the linear readouts; and
apply the machine learning model to a target linear readout representing at least a portion of the first amino acid along a target peptide, to identify the target peptide.

In some embodiments, the method is for identifying a peptide. In some embodiments, the system is for use in identifying a peptide. As used herein, the term “identifying” does not require providing the full sequence of a peptide, but rather identifying it by name. Proteins often have multiple isoforms or point mutations and the method of the invention need not provide the full sequence of an analyzed peptide but rather merely identify the protein by name so as to distinguish it from other proteins. Similarly, a protein may be identified as being a protein in a group of proteins, such as the protein is either protein A or protein B. It is often useful to know the proteomic make up of a sample, even if the specific isoforms or sequences of the proteins in the sample do not need to be known. Thus, for example a protein being analyzed could be identified as “Albumen” even if the full sequence of albumen is not detected.

In some embodiments, the method is for sequencing a peptide. In some embodiments, the system is for identifying a peptide. In some embodiments, the method is for identifying a plurality of peptides in a sample. In some embodiments, the method if for identifying a purified peptide. In some embodiments, the method is for proteomic analysis. In some embodiments, the method is for proteomic analysis of a sample. In some embodiments, the method is for peptide quantification. In some embodiments, the method is for relative peptide quantification. In some embodiments, the method is for distinguishing a peptide from other peptides in a set of peptides.

As used herein, the terms “peptide”, “polypeptide” and “protein” are used interchangeably to refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides polypeptides and proteins described have modifications rendering them more stable while in the body or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

As used herein, the term “isolated peptide” refers to a peptide that is essentially free from contaminating cellular components, such as carbohydrate, lipid, or other proteinaceous impurities associated with the peptide in nature. Typically, a preparation of isolated peptide contains the peptide in a highly purified form, i.e., at least about 80% pure, at least about 90% pure, at least about 95% pure, greater than 95% pure, or greater than 99% pure.

In some embodiments, the peptide is a protein. In some embodiments, the peptide is an isolated peptide. In some embodiments, the peptide is a peptide from a sample. In some embodiments, the peptide is a complete protein. In some embodiments, the peptide is an intact protein. In some embodiments, the peptide is an undigested protein. In some embodiments, the peptide is an unfragmented protein. In some embodiments, the peptide is a protein that has not been shortened artificially. In some embodiments, artificially is in vitro. In some embodiments, the peptide is a fragment of a protein. In some embodiments, the peptide is at least 50, 55, 60, 65, 70, 75, 80, 85, 90, 95, 97, 99 or 100% of a protein. Each possibility represents a separate embodiment of the invention. In some embodiments, the peptide is a native protein. In some embodiments, the peptide is a naturally occurring peptide. In some embodiments, the peptide is not a cleaved peptide. In some embodiments, the peptide is not a digested peptide. In some embodiments, the peptide is not produced by cleaving or digesting an intact protein.

In some embodiments, the peptide comprises at least 2, 3, 5, 10, 15, 20, 25, 30, 35, 40, 45, 50, 55, 60, 65, 70, 75, 80, 85, 90, 95, 100, 125, 150, 175, 200, 250, 300, 350, 400, 450, 500, 600, 700, 800, 900, 1000, 1250, 1500, 1750, 2000, 2500, or 3000, amino acids. Each possibility represents a separate embodiment of the invention. In some embodiments, the peptide comprises at least 1, 2, 3, 4, 5, 6, 7, 8, 9, 10, 12, 15, 17, 20 or 25 of the first amino acid. Each possibility represents a separate embodiment of the invention.

In some embodiments, the readout is embodied in an electronic file. In some embodiments, the readout is an electronic file. In some embodiments, the readout is further representative of at least a portion of a second amino acid along the peptide. In some embodiments, the readout is further representative of at least a portion of a third amino acid along the peptide. In some embodiments, the readout is representative of at least a portion of 1, 2, 3, 4, or 5 amino acids along the peptide. Each possibility represents a separate embodiment of the invention.

It will be understood by a skilled artisan that when referring herein to a first amino acid and a second amino acid reference is being made to different types or species of amino acids and not single individual amino acids along a chain. Thus, a first amino acid might be, for example, lysine; and a second amino acid might be, for example, cysteine. In some embodiments, the first, second, third or any amino acid recited herein is a specific amino acid species. As used herein, the term “amino acid species” refers to any specific amino acid, such as lysine, cysteine, methionine, alanine, histidine etc. In some embodiments, the first, second, third or any amino acid recited herein is a type of amino acid. In some embodiments, a type of amino acid refers to group of amino acids with a common structure or characteristic. Types of amino acids include, but are not limited to, aromatic amino acids, non-polar amino acids, charged amino acids, and polar amino acids. In some embodiments, an amino acid is a naturally occurring amino acid. In some embodiments, an amino acid comprises artificial amino acids. In some embodiments, the amino acid is a mammalian amino acid. In some embodiments, the mammal is human. In some embodiments, an amino acid is selected from: aspartic acid, threonine, serine, glutamic acid, proline, glycine, alanine, valine, cysteine, methionine, isoleucine, leucine, tyrosine, phenylalanine, lysine, histidine, arginine, tryptophan asparagine, and glutamine.

In some embodiments, the amino acid is an amino acid that can be uniquely labeled. It will be understood by a skilled artisan that while the labeling of three specific amino acids (lysine, cysteine and methionine) is embodied in the examples section hereinbelow, such illustration is merely by way of example. Lysine, cysteine and methionine can be uniquely labeled by separate chemistries and thus can be analyzed together. Use of another three amino acids or a combination of only 1 or 2 of the exemplified amino acids with other amino acids that can be uniquely labeled would result in a similar analysis. Even a labeling with less specificity, such as a label that marks two amino acids uniquely, can be employed. Similarly, higher combinations, mixes or four unique labels or five unique labels will work on the same principle and may allow for more rapid identification, or identification with worse resolution. In some embodiments, the first and second amino acids are different amino acids. In some embodiments, the first, second and third amino acids are different amino acids. In some embodiments, the first and any subsequent amino acids are different amino acids. In some embodiments, different amino acids can be differentially and/or uniquely labeled. Examples of unique amino acid labeling include, but are not limited to, labeling the thiol group of cysteine, labeling the amine group of lysine, labeling the sulfur of methionine, labeling the indole side chain of tryptophan, labeling the phenolic side chain of tyrosine, and labeling the glutamyl/aspartyl side chains of glutamic acid and aspartic acid. Commercial kits for such labeling are known in the art and include, but are not limited to, the STELLA+lysine labeling kit, the Monolith NHS kit (amine reactive), and the Monolith Maleimide kit (cysteine reactive). Additionally, artificial amino acids may be used during protein/peptide synthesis such that the artificial amino acids may be specifically labeled. Similarly, natural amino acids may be post-translationally modified to generate a moiety for specific labeling.

In some embodiments, the readout is a linear readout. A linear readout refers to a presentation of the amino acids as they appear in the sequence of the peptide, if the peptide is viewed linearly as a single string of amino acids. The linearity of the peptide can be considered from its N-terminus to C-terminus or in the reverse. Either direction is still considered linear. In some embodiments, the readout is from N-terminus to C-terminus. In some embodiments, the readout is from C-terminus to N-terminus. In some embodiments, the readout is from N-terminus to C-terminus or C-terminus to N-terminus. In some embodiments, the linear readout is representative of the order of amino acids along the peptide. In some embodiments, the linear readout is representative of the relative position of the amino acids along the peptide. In some embodiments, the readout is representative of the linear pattern of the amino acid. In some embodiments, the readout is a low-resolution linear pattern of the amino acid. In some embodiments, the readout is a low-resolution linear positioning of the amino acid along the peptide. In some embodiments comprising representation of more than one amino acid, the linear readout represents relative information on the order and/or position of the more than one amino acids.

In some embodiments, the first amino acid is selected from lysine, cysteine and methionine. In some embodiments, the second amino acid is selected from lysine, cysteine and methionine. In some embodiments, the third amino acid is selected from lysine, cysteine and methionine. In some embodiments, the first, second and third amino acids are lysine, cysteine and methionine.

As used herein, “a portion” of an amino acid refers to at least one of all of the particular amino acids along the peptide. A peptide may have many residues of one particular amino acid, and a portion refers to at least one of those residues. In some embodiments, a portion is at least 20, 25, 30, 35, 40, 45, 50, 55, 60, 65, 70, 75, 80, 85, 90, 95, 97, 99 or 100% of all residues of the amino acid along the peptide. Each possibility represents a separate embodiment of the invention. In some embodiments, a portion is at least 60%. In some embodiments, a portion is at least 70%. In some embodiments, a portion is at least 80%. In some embodiments, a portion is at least 90%. In some embodiments, a portion is not 100%. In some embodiments, a portion does not comprise 100%. It will be understood by a skilled artisan that not every portion must be the same percentage. For example, labeling of a first amino acid may be less efficient than labeling of a second amino acid, and therefore the portion of the first amino acid may be smaller than the portion of the second amino acid. Similarly, for any other conditions that may affect the size of the portion represented in the readout, it need not be such that each amino acid be represented by the same size portion or by the same number of amino acid residues.

As will be understood by a skilled artist, specific methods of labeling of amino acids have varying labeling efficiencies depending on the method of labeling and the target amino acid. Because this inefficiency in labeling is generally unbiased, different residues of a peptide may be labeled each time a given peptide is labeled. Further, most label scanning/detecting technologies also lack 100% accuracy and thus correctly labeled amino acids may be missed or not detected. Similarly, depending on the resolution of the scanning device, two labeled amino acids that are in close proximity may not be uniquely detected, and/or their relative position may not be identifiable. The resolution may also depend on other factors such as the velocity of the peptide as it is being scanned, the medium in which it is being scanned (viscosity, electrical properties, etc.) and the general physical conditions (pH, temp, etc.) during scanning. All of these issues may lead to an imperfect readout in which not every amino acid that should be detected is, but rather only a portion of the amino acids are present in the readout. The methods of the invention are unexpectedly useful in that even with such degenerate readouts for a peptide, the peptides true identity can be accurately assessed.

In some embodiments, the machine learning model is a machine learning classifier. In some embodiments, the machine learning model is a machine learning algorithm. In some embodiments, the algorithm is a supervised learning algorithm. In some embodiments, the algorithm is an unsupervised learning algorithm. In some embodiments, the algorithm is a reinforcement learning algorithm. In some embodiments, the machine learning model is a Convolutional Neural Network (CNN).

In some embodiments, the machine learning model predicts the identity of the peptide. In some embodiments, the machine learning model outputs the identity of the peptide. In some embodiments, the machine learning model predicts the sequence of the peptide. In some embodiments, the machine learning model predicts with at least 70, 75, 80, 85, 90, 95, 97, 99 or 100% accuracy. Each possibility represents a separate embodiment of the invention. In some embodiments, the machine learning model predicts at most 2 possibilities for the identity of the peptide. In some embodiments, the machine learning model further outputs a confusion matrix for the peptide. In some embodiments, the confusion matrix indicates the probability for correct identification.

In some embodiments, the machine learning model is trained on readouts of a set of peptides. In some embodiments, the machine learning model is trained on a training set of readouts. In some embodiments, the peptide to be identified is in the set of peptides. In some embodiments, the peptide to be identified is predicted to be in the set of peptides. In some embodiments, the readouts of the training set represent at least a portion of the first amino acid along a peptide from the set of peptides. In some embodiments, the readouts of the training set represent at least a portion of 1, 2, 3, 4, or 5 amino acids along the peptide from the set of peptides. In some embodiments, the readouts of the training set represent at least a portion of the first amino acid and a portion of the second amino acid and optionally a portion of the third amino acid along the peptide from the set of peptides.

In some embodiments, the set of peptides is a set of peptides with known sequences. In some embodiments, the set of peptides is a set of peptides with known readouts. In some embodiments, the set of peptides is a set of peptides expected to be in a sample. In some embodiments, the peptide to be analyzed in from the sample. In some embodiments, the sample is a bodily fluid. In some embodiments, a bodily fluid is selected from at least one of blood, plasma, serum, tissue, urine, gastric fluid, intestinal fluid, saliva, bile, tumor fluid, breast milk, interstitial fluid, stool and cerebral spinal fluid. In some embodiments, the sample is a biopsy. In some embodiments, the biopsy is a liquid biopsy. In some embodiments, the sample is protein panel. Protein panels are well known in the art, such as, for non-limiting example, a cytokine panel, oncogene panel, surface marker panel and a clinical biomarker panel.

In some embodiments, the set of peptides are the proteins found in a proteome. In some embodiments, the proteome is full organism proteome. In some embodiments, the organism is a mammalian. In some embodiments, the mammal is a human. In some embodiments, the peptide to be analyzed is from the proteome. In some embodiments, the set of peptides are proteins found in a bodily fluid. In some embodiments, the peptide to be analyzed is in the bodily fluid. In some embodiments, the proteome is an organ, tissue or fluid proteome. In some embodiments, the fluid is a bodily fluid. In some embodiments, the tissue is tumor tissue. In some embodiments, the tissue is a tumor. In some embodiments, the set pf proteins are proteins found in plasma. In some embodiments, the protein to be analyzed is from plasma.

In some embodiments, the set of proteins comprises at least 2, 5, 7, 10, 12, 15, 20, 25, 30, 40, 50, 100, 200, 300, 400, 500, 600, 700, 800, 900, 1000, 5000, 10000, 15000, 20000, or 25000 proteins. Each possibility represents a separate embodiment of the invention.

The sequences of proteins that may be used for generation of simulated traces are easily accessible to one skilled in the art. For example, amino acid sequences can be found in the Pubmed, Uniprot and Swissprot databases. Additionally, the expected protein makeup of whole organism genomes are also available on these databases. Further, the proteome or expected proteome for various tissues and fluids can be found, for example, at the Human Protein Atlas, or the Tissues database, as well as at the above databases that provide whole proteome data.

In some embodiments, the analyzed readout is the same type of readout as the readouts of the training set. In some embodiments, the training set comprises a plurality of readouts. In some embodiments, each readout represents at least a portion of a first amino acid along a peptide. In some embodiments, each readout represents at least a portion of a second amino acid along a peptide. In some embodiments, each readout represents at least a portion of a third amino acid along a peptide. In some embodiments, each readout represents at least a portion of a fourth amino acid along a peptide. In some embodiments, each readout represents at least a portion of a fifth amino acid along a peptide.

In some embodiment, the training set comprises at least 10, 15, 20, 25, 30, 40, 50, 60, 70, 75, 80, 90, 100, 150, 200, 250, 300, 350, 400, 450, 500, 600, 700, 800, 900, or 1000 readouts representative of a peptide. Each possibility represents a separate embodiment of the invention. In some embodiments, the training set comprises at least 2, 5, 7, 10, 12, 15, 20, 25, 30, 40, 50, 100, 200, 300, 400, 500, 600, 700, 800, 900, 1000, 5000, 10000, 15000, 20000, or 25000 proteins. Each possibility represents a separate embodiment of the invention. In some embodiments, the training set comprises at least 10, 15, 20, 25, 30, 40, 50, 60, 70, 75, 80, 90, 100, 150, 200, 250, 300, 350, 400, 450, 500, 600, 700, 800, 900, or 1000 readouts representative of each peptide from the set. Each possibility represents a separate embodiment of the invention.

In some embodiments, the training set comprises labels identifying the peptide associated with each readout. In some embodiments, the training set comprises labels identifying the peptide represented in each readout. In some embodiments, the training set comprises labeled readouts, wherein the label identifies the peptide associated with the readout. In some embodiments, the training set comprises labeled readouts, wherein the label identifies the peptide represented in the readout.

In some embodiments, the readouts of the training set comprise at least 10, 15, 20, 25, 30, 40, 50, 60, 70, 75, 80, 90, 100, 150, 200, 250, 300, 350, 400, 450, 500, 600, 700, 800, 900, or 1000 readouts representative of a peptide from the set. Each possibility represents a separate embodiment of the invention. In some embodiments, the readouts of the training set comprise at least 50 readouts representative of a peptide from the set. In some embodiments, the readouts of the training set comprise at least 80 readouts representative of a peptide from the set. In some embodiments, the readouts of the training set comprise at least 10, 15, 20, 25, 30, 40, 50, 60, 70, 75, 80, 90, 100, 150, 200, 250, 300, 350, 400, 450, 500, 600, 700, 800, 900, or 1000 readouts representative of each peptide from the set. Each possibility represents a separate embodiment of the invention. In some embodiments, the readouts of the training set comprise at least 50 readouts representative of each peptide from the set. In some embodiments, the readouts of the training set comprise at least 80 readouts representative of each peptide from the set.

In some embodiments, the readouts of the training set are simulated readouts. In some embodiments, the training set comprises simulated readouts. In some embodiments, the simulated readouts are based on a known sequence for a peptide. In some embodiments, the simulated readouts are based on a known sequence for each peptide. In some embodiments, the simulations are generated with a non-ideal condition. In some embodiments, the condition is selected from non-ideal labeling efficiency and non-ideal detection resolution. In some embodiments, the condition is selected from non-ideal labeling efficiency, non-ideal detection resolution, and non-ideal conditions during detection. In some embodiments, non-deal conditions during detection are selected from non-ideal pH, non-ideal temperature, non-ideal speed of the peptide. In some embodiments, the condition is selected from non-ideal labeling efficiency, non-ideal detection resolution, and non-deal velocity of the peptide as it is detected. In some embodiments, the simulations are based on a known sequence when only a portion of an amino acid is represented in the simulated readout. In some embodiments, the simulations are based on a known sequence when at least a portion of an amino acid is not represented in the simulated readout.

It will be understood, that given a known sequence of a protein, simulated readouts can be generated with only a certain percentage of labeling or only with a given spatial resolution or generally with any desired constraint. Several readouts for each condition can be generated, as labeling only 80% of an amino acid for example, can lead to numerous permutations of a simulated readout. For an illustrative example, if a peptide comprises four lysine residues {K1, K2, K3 and K4}, a 75% labeling can result in 4 different possibilities: {K1, K2, K3}, {K1, K2, K4}, {K1, K3, K4} and {K2, K3, K4}. In some embodiments, the training set comprises simulation of every possibility for a given condition. In some embodiments, the training set comprises at least 5, 10, 15, 20, 25, 30, 40, 50, 60, 70, 75, 80, 90, 95, 97, 99 or 100% of every possibility for a given condition. Each possibility represents a separate embodiment of the invention. In some embodiments, the training set comprises a plurality of simulated condition. In some embodiments, the training set comprises at least 1, 2, 3, 4, 5, 6, 7, 8, 9 or 10 simulated conditions. Each possibility represents a separate embodiment of the invention.

In some embodiments, the method further comprises receiving a readout representative of the peptide to be analyzed. In some embodiments, the method further comprises receiving a readout representative of a target peptide. In some embodiments, a target peptide is a peptide to be analyzed. In some embodiments, the target peptide is a peptide in a sample. In some embodiments, the target peptide is a peptide expected to be in a sample. In some embodiments, the target peptide is in the sample. In some embodiments, the target peptide is from the sample. In some embodiments, the method further comprises an inference stage. In some embodiments, the inference stage comprises applying the machine learning model to a target readout. In some embodiments, the machine learning model is the trained machine learning model. In some embodiments, the target readout represents at least a portion of a first amino acid along a target peptide. In some embodiments, the target readout represents at least a portion of a second amino acid along a target peptide. In some embodiments, the target readout represents at least a portion of 1, 2, 3, 4 or 5 amino acids along a target peptide. Each possibility represents a separate embodiment of the invention.

In some embodiments, the method further comprises receiving a readout representative of at least a portion of a first amino acid along a peptide. In some embodiments, the received readout is a linear readout. In some embodiments, the received readout is of at least a portion of a first amino acid and at least a portion of a second amino acid and optionally at least a portion of a third amino acid, fourth amino acid or fifth amino acid along the peptide.

In some embodiments, the method further comprises labeling at least a portion of an amino acid with a label along the peptide. In some embodiments, the received readout and/or the readout to be analyzed is generated by labeling at least a portion of an amino acid with a label along the peptide. In some embodiments, the amino acid is the first amino acid and the label is a first label. In some embodiments, the amino acid is the second amino acid and the label is a second label. In some embodiments, the amino acid is the third amino acid and the label is a third label. In some embodiments, each different amino acid is labeled with a different label. Thus, if three amino acids are to be part of the readout then those three amino acids are labeled each with a distinct label.

In some embodiments, the method further comprises detecting the labels linearly along the peptide. In some embodiments, the detecting the labels linearly along the peptide is to produce the readout. In some embodiments, the received readout and/or the readout to be analyzed are produced by detecting the labels linearly along the peptide. In some embodiments, detecting linearly comprises detecting the order along the peptide. In some embodiments, the detecting linearly comprises detecting the relative order of more than one amino acid along the peptide. In some embodiments, detecting linearly comprises detecting a low-resolution pattern of the amino acid along the peptide. In some embodiments, detecting linearly comprises detecting the low-resolution position of the amino acid along the peptide. In some embodiments, all labeled amino acids are detected. In some embodiments, at least 1, 2, 3, 4, or 5 labeled amino acids are detected. Each possibility represents a separate embodiment of the invention.

In some embodiments, each labeled amino acid along the peptide is detected. In some embodiments, at least 50, 55, 60, 65, 70, 75, 80, 85, 90, 95, 97, 99 or 100% of the labeled amino acids along the peptide are detected. Each possibility represents a separate embodiment of the invention. Depending on the resolution of the detecting device not all labels may be uniquely detected. Further, the experimental conditions during detection may result in non-ideal detection causing either missing of a label or incorrect ordering of a label.

In some embodiments, the detecting comprises passing the labeled peptide through a nanopore. In some embodiments, a label is uniquely detectable as it passes through the nanopore. In some embodiments, the nanopore comprises a sensor. In some embodiments, the nanopore is coupled to a sensor. In some embodiments, the sensor is configured for detection of the label. In some embodiments, the sensor is configured for detection at the nanopore. In some embodiments, the sensor is configured for detection at the exit of the nanopore. In some embodiments, the sensor is configured for detection of the label at the nanopore or at the exit of the nanopore. In some embodiments, each label is uniquely detectable as it passes through the nanopore. In some embodiments, a label comprises a fluorophore or a fluorescent moiety. In some embodiments, the nanopore comprises or is coupled to an optical sensor. In some embodiments, the optical sensor is configured to detect fluorescence at the nanopore. In some embodiments, the optical sensor is configured to detect fluorescence at the exit of the nanopore. In some embodiments, a label comprises a bulky group. In some embodiments, the nanopore comprises or is coupled to an electrical sensor. In some embodiments, electrical sensor is configured to detect electrical current at the nanopore. In some embodiments, the electrical sensor is configured to detect electrical voltage at the nanopore. In some embodiments, the electrical sensor is configured to detect electrical current, voltage or both at the nanopore.

Different fluorochromes have distinct excitation ranges and emission ranges allowing for unique detection by a single sensor or by a plurality of sensors. In some embodiments, a dedicated sensor detects each label. These fluorochromes and their excitation and emission ranges are well known in the art. Some non-limiting examples of fluorochromes and their maximum excitation and emission wavelengths (nm) include: 7-AAD (7-Aminoactinomycin D) 546, 647; Acridine Orange (+DNA) 500, 526; Acridine Organe (+RNA) 460, 650; Allophycocyanin (APC) 650, 660; Aniline Blue 370, 509; BODIPY® FL 505, 513; CF640R 642, 662; Cy5® 649, 670; Cy5.5® 675, 694; Cy7® 743, 767; DAPI 358, 461; EGFP 489, 508; Fluorescein (FITC) 494, 518; Pacific Blue 410, 455; PE (R-phycoerythrin) 480 and 565, 575; PE-Cy5480 and 650, 670; PE-Cy7480 and 743, 767; Propidium Iodide (PI) 536, 617; and YFP (Yellow Fluorescent Protein) 513, 527. Spectra for fluorochromes can also be found at the following websites: probes.com/servlets/spectra/and clontech.com/gfp/excitation.shtml as well as many others known to those skilled in the art. Detection of each

According to some embodiments, the nanopore is an ion-conducting nanopore. In some embodiments, the nanopore is a solid-state nanopore. In some embodiments, the nanopore is a plasmonic nanopore. In some embodiments, the nanopore is a plasmonic nanowell.

In some embodiments, the nanopore is part of a nanopore apparatus. In some embodiments, the nanopore is in a film. The production of nanopores in a film is well known in the art. Fabrication of nanopores in thin membranes has been shown in, for example, Kim et al., Adv. Mater. 2006, 18 (23), 3149 and Wanunu, M. et al., Nature Nanotechnology 2010, 5 (11), 807-814. Further, methods of such fabrication of films in silicon wafers, and methods of producing nanopores therein are provided herein in the Materials and Methods section. In some embodiments, the nanopore is produced with a transition electron microscope (TEM). In some embodiments, the nanopore is produced with a high-resolution aberration-corrected TEM or a noncorrected TEM.

According to some embodiments, the nanopore apparatus comprises a film, and wherein the film comprises at least one nanopore. In some embodiments, the nanopore apparatus further comprises a first and a second fluidic reservoir separate by the film and connected via the nanopore. In some embodiments, the nanopore apparatus further comprises first and second electrodes configured to electrically contact fluid placed in the first reservoir and fluid placed in the second reservoir, respectively. In some embodiments, the electrodes are configured to generate an electrical current that drives a protein to be analyzed through the nanopore.

In some embodiments, the nanopore is naked in that it does not comprise a protein for facilitating transfer through the nanopore. In some embodiments, the labeled protein passes through the nanopore via the electrical current generated by the electrodes. In some embodiments, the labeled protein is denatured. In some embodiments, the protein is denatured with a surfactant. In some embodiments, the surfactant is sodium dodecyl sulfate (SDS). In some embodiments, the labeled protein is uniformly labeled by a charge to induce transfer through the nanopore. In some embodiments, the charge is a negative charge. In some embodiments, the nanopore apparatus further comprises a sensor or detector for detecting a label as it passes through the nanopore. In some embodiments, the label is detected at the nanopore. In some embodiments, the label is detected at the exit of the nanopore. In some embodiments, the label is detected while exiting the nanopore.

In some embodiments, the readout is a linear trace of the peptide as it passes through the nanopore. In some embodiments, the linear trace is a linear-temporal trace. In some embodiments, the readout represents the time of each label along the peptide as it passes through the nanopore. In some embodiments, the time of passage is roughly proportional to position along the peptide. It will be understood by a skilled artisan that different amino acids will pass through a naked nanopore at different speeds and with different translocation rates. Since the movement is not linear, the temporal trace does not perfectly correlate to positions along the peptide, although a low-resolution positioning can be discerned. Although precise positioning is not known, the time traces can be analyzed by the machine learning model to better distinguish between peptides with similar orders of labeled amino acids, but with different positions temporally. In some embodiments, linear-temporal traces are used for training the machine learning model.

In some embodiments, the nanopore comprises a diameter not greater than 1, 2, 3, 4, 5, 7, 10, 15, 20, 15, 30, 35, 40, 45, 50, 55, 60, 65, 70, 75, 80, 85, 90, 95, 100, 110, 120, 130, 140, 150 nm. Each possibility represents a separate embodiment of the invention. In some embodiments, the nanopore comprises a diameter not greater than 5 nm. In some embodiments, the nanopore comprises a diameter not greater than 7 nm. In some embodiments, the nanopore comprises a diameter not greater than 100 nm. In some embodiments, the nanopore comprises a diameter of about 5 nm. In some embodiments, the nanopore comprises a diameter between 0.5 and 5, 0.5 and 7, 0.5 and 10, 0.5 and 15, 0.5 and 20, 1 and 5, 1 and 7, 1 and 10, 1 and 15, 1 and 20, 3 and 5, 3 and 7, 3 and 10, 3 and 15, 3 and 20, 5 and 7, 5 and 10, 5 and 15, or 5 and 20 nm. Each possibility represents a separate embodiment of the invention. The width of an amino is ˜2 nm and the Kuhn length for a polypeptide is ˜7 nm, therefore nanopores in this size range are ideal. However, as demonstrated hereinbelow, even far worse spatial resolution can still be used as part of the method of the invention.

In some embodiments, the nanopore comprises a resolution not greater than 1, 2, 3, 4, 5, 7, 10, 15, 20, 15, 30, 35, 40, 45, 50, 55, 60, 65, 70, 75, 80, 85, 90, 95, 100, 110, 120, 130, 140, 150 nm. Each possibility represents a separate embodiment of the invention. In some embodiments, the nanopore comprises a resolution not greater than 5 nm. In some embodiments, the nanopore comprises a resolution not greater than 7 nm. In some embodiments, the nanopore comprises a resolution not greater than 100 nm. In some embodiments, the nanopore comprises a resolution of about 5 nm. In some embodiments, the nanopore comprises a resolution between 0.5 and 5, 0.5 and 7, 0.5 and 10, 0.5 and 15, 0.5 and 20, 1 and 5, 1 and 7, 1 and 10, 1 and 15, 1 and 20, 3 and 5, 3 and 7, 3 and 10, 3 and 15, 3 and 20, 5 and 7, 5 and 10, 5 and 15, or 5 and 20 nm. Each possibility represents a separate embodiment of the invention.

In some embodiments, the nanopore comprises a plasmonic structure. In some embodiments, the structure is a nano-structure. Such nanopores are known in the art as plasmonic nanopores. In some embodiments, the plasmonic structure is configured to localize electromagnetic excitation below a wavelength of light. In some embodiments, the wavelength below a wavelength of light is a particular wavelength. In some embodiments, the particular wavelength is a wavelength of the fluorescent label to be detected. In some embodiments, the plasmonic structure is configured to amplify localized fluorescence emission at the nanopore. In some embodiments, the amplification is at a plurality of wavelengths. In some embodiments, the amplification is at a particular wavelength. In some embodiments, the plurality of wavelengths comprise wavelengths of the fluorochrome labels.

The plasmonic nanopores and nanowells can be configured to enhance specific excitation and thereby specific flourochromes. Configurations of nanowells to enhance excitation at specific or multiple plasmonic resonances are well known in the art and comprise using particular geometries, dimensions, materials, refractive indecies or a combination thereof. Examples of these geometries, materials and dimensions can be found in Fermamdez-Garcia, et al., Design Considerations for Near-filed Enhancement in Optical Antennas, Contemporary Physics, 2014, and may include for example rod, ellipsoid, bowtie, disk and square geometries; gold, silver aluminum and copper nanowells; as well as diameters measuring about 40, 30, 20, 10 and 5 nm. Configurations of plasmonic nanopores and methods of producing plasmonic nanopores can be found in International Patent Publication WO2019/123467, which is herein incorporated by reference in its entirety.

In some embodiments, the method can be for identifying a plurality of peptides in a sample. In some embodiments, readouts from the plurality of peptides are analyzed. In some embodiments, the sample is passed through the nanopore and the peptides are analyzed. In some embodiments, the sample is provided to the first reservoir of the nanopore apparatus and the peptides are detected to produce readouts for each protein. In some embodiments, the apparatus comprises an array of nanopores so that a plurality of peptides is detected simultaneously.

As used herein, the terms “electronic document” and “electronic file” are interchangeable and refer broadly to any document/file containing data and stored in a computer-readable format. Electronic document formats may include, among others, Portable Document Format (PDF), Digital Visual Interface (DVI), text files (txt), Comma Separated Vector (CSV), binary files, NumPy array files (npy), PostScript, word processing file formats, such as docx, doc, and Rich Text Format (RTF), and/or XML Paper Specification (XPS).

In some embodiments, the labels denote the identity of the peptide. In some embodiments, the labels identify the peptide by name. In some embodiments, the labels are the name of the peptide. In some embodiments, the labels are the protein abbreviate of the name of the protein. For example, the abbreviate for Albumen is known in the art to be ALB. In some embodiments, the labels are database numbers for the proteins. In some embodiments, the labels are sequences of the proteins. In some embodiments, the labels are tags for the proteins.

In some embodiments, the one or more new documents/file contain readouts from a peptide to be identified. In some embodiments, the one or more new documents/files contain readouts from a peptide from a sample. In some embodiments, the training set comprises readouts of a set of peptides in, or expected to be in, the sample. In some embodiments, the training set comprises readouts of proteins found in a proteome. In some embodiments, the training set comprises readouts of all proteins found in a proteome. In some embodiments, the training set comprises readouts for at least 2, 5, 7, 10, 12, 15, 16, 17, 18, 19, 20, 25, 30, 40, 50, 100, 200, 300, 400, 500, 600, 700, 800, 900, 1000, 5000, 10000, 15000, 20000, or 25000 proteins. Each possibility represents a separate embodiment of the invention. In some embodiments, the training set comprises readouts for at least 15 proteins. In some embodiments, the training set comprises readouts for at least 16 proteins. In some embodiments, the training set comprises readouts for at least 50 proteins. In some embodiments, the training set comprises at least 20, 25, 30, 40, 50, 60, 70, 75, 80, 90, 100, 150, 200, 250, 300, 350, 400, 450, 500, 600, 700, 800, 900, or 1000 readouts representative of a peptide from the set. Each possibility represents a separate embodiment of the invention. In some embodiments, the training set comprises at least 20, 25, 30, 40, 50, 60, 70, 75, 80, 90, 100, 150, 200, 250, 300, 350, 400, 450, 500, 600, 700, 800, 900, or 1000 readouts representative of each peptide from the set. Each possibility represents a separate embodiment of the invention. In some embodiments, the training set comprises at least 50 readouts representative of a peptide from the set. In some embodiments, the training set comprises at least 50 readouts representative of each peptide from the set. In some embodiments, the training set comprises at least 80 readouts representative of a peptide from the set. In some embodiments, the training set comprises at least 80 readouts representative of each peptide from the set.

In some embodiments, the one or more new electronic documents are one new document. In some embodiments, the one or more new electronic documents are a plurality of documents. In some embodiments, the one or more new electronic documents are proteins from a sample. In some embodiments, the one or more new electronic documents comprise a readout of a peptide to be analyzed. In some embodiments, the one or more new electronic documents comprise a readout of a peptide from a sample. In some embodiments, the one or more new electronic documents comprise a readout of a peptide as it passes through a nanopore. In some embodiments, the one or more new electronic documents comprise a linear temporal trace of a labeled peptide as it passes through a nanopore.

In some embodiments, the labeled peptide is labeled at at least a portion of one amino acid. In some embodiments, the labeled peptide is labeled at at least a portion of a plurality of amino acids. In some embodiments, the labeled peptide is labeled at at least a portion of 1, 2, 3, 4, 5, 6, 7, 8, 9 or 10 amino acids. Each possibility represents a separate embodiment of the invention. In some embodiments, the labeled peptide is labeled at at least a portion of two amino acids. In some embodiments, the labeled peptide is labeled at at least a portion of three amino acids. In some embodiments, the amino acids are the first, second, third amino acid or a combination thereof.

In some embodiments, the at least one hardware processor trains a machine learning model. In some embodiments, the model is based, at least in part, on a training set. In some embodiments, the model is based on a training set. In some embodiments, the at least one hardware processor applies the machine learning model to a target readout. In some embodiments, the target readout is a linear readout. In some embodiments, the target readout represents at least a portion of a first amino acid along a target peptide. In some embodiments, the target readout represents at least a portion of a second amino acid along a target peptide. In some embodiments, the target readout represents at least a portion of a third amino acid along a target peptide. In some embodiments, the target readout represents at least a portion of 1, 2, 3, 4 or 5 amino acids along a target peptide. Each possibility represents a separate embodiment of the invention.

According to some embodiments, the system further comprises means for producing the plurality of electronic documents. In some embodiments, the system further comprises a nanopore. In some embodiments, the system further comprises a nanopore apparatus. In some embodiments, the means for producing the plurality of electronic documents is the nanopore apparatus.

In some embodiments, the present invention may be configured for automatic document classification based, at least in part, on content-based assignment of one or more predefined categories (classes) to documents. By classifying the content of a document, it may be assigned one or more predefined classes or categories, thus making it easier to manage and sort. Such classes may be specific families of proteins, proteins with particular functions, proteins from particular sources or any class of protein or category of protein such as would be useful to the user.

Typically, multi-class machine learning classifiers are trained on a training set of documents, where each document belongs to one of a certain number of distinct classes (e.g., invoices, scientific papers, resumes, letters). The training set may be labeled with the correct classes (e.g., for supervised learning), or may not be labeled (e.g., in the case of unsupervised learning). Following a training stage, the classifier may be able to predict the most probable class for each document in a test set of documents. Although document classification may be based on textual content alone, for some types of documents, the task of classification can be significantly enhanced by also generating features from the visual structure of the document. This is based on the idea that documents in the same category often also share similar layout and structure features.

In some embodiments, following a multi-modal training stage, a trained classifier of the present invention may be configured for classifying electronic documents based on a multi-modal input comprising both representations of the documents. In other embodiments, the trained classifier may be configured for classifying electronic documents based on only a single modality input (e.g., textual content or raster image alone), with improved classification accuracy as compared to a classifier which has been trained solely based on a single modality.

In some embodiments, the present invention may employ one or more types of neural networks to further generate data representations of the multi-modal inputs. For example, raw input text from an electronic document may be processed so as to generate a data representation of the text as a fixed-length vector. Similarly, images of the electronic document (e.g., thumbnails or raster images) may be processed to extract image features.

In some embodiments, the neural network models employed by the present invention to generate textual data representations may be selected from the group consisting of Neural Bag-of-Words (NBOW); recurrent neural network (RNN), Recursive Neural Tensor Network (RNTN); Dynamic Convolutional Neural Network (DCNN); Long short-term memory network (LSTM); and recursive neural network (RecNN). See, e.g., Pengfei Liu et al., “Recurrent Neural Network for Text Classification with Multi-Task Learning”, Proceedings of the Twenty-Fifth International Joint Conference on Artificial Intelligence (IJCAI-16). Convolutional neural network (CNN) may be used, e.g., to extract image features which represent the physical visual structure of a document.

In some embodiments, the present invention may further be configured for employing a common representation learning (CRL) framework, for learning a common representation of the two views of data (i.e., textual and visual). CRL is associated with multi-view data that can be represented in multiple forms. The learned common representation can then be used to train a model to reconstruct all the views of the data from each input. CRL of multi-view data can be categorized into two main categories: canonical-based approaches and autoencoder-based methods. Canonical Correlation Analysis (CCA)-based approaches comprise learning a joint representation by maximizing correlation of the views when projected to the common subspace. Autoencoder (AE) methods learn a common representation by minimizing the error of reconstructing the two views. AE-based approaches use deep neural networks that try to optimize two objective functions. The first objective is to find a compressed hidden representation of data in a low-dimensional vector space. The other objective is to reconstruct the original data from the compressed low-dimensional subspace. Multi-modal autoencoders (MAE) are two-channeled models which specifically perform two types of reconstructions. The first is the self-reconstruction of view from itself, and the other is the cross-reconstruction where each view is reconstructed from the other. These reconstruction objectives provide MAE the ability to adapt towards transfer learning tasks as well. In the context of CRL, each of these approaches has its own advantages and disadvantages. For example, though CCA based approaches outperform AE based approaches for the task of transfer learning, they are not as scalable as the latter.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Rather, the computer readable storage medium is a non-transient (i.e., not-volatile) medium.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### Examples

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Methods

### A Theoretical Analysis of the Proteins ID Based on 2 or 3 Amino-Acid Tags

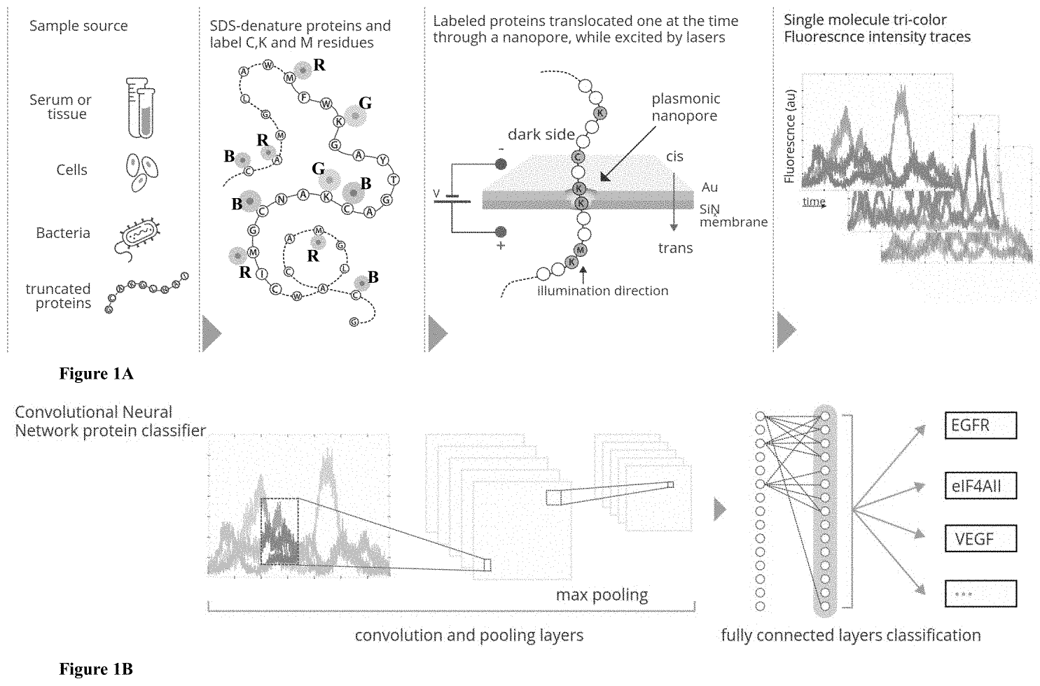

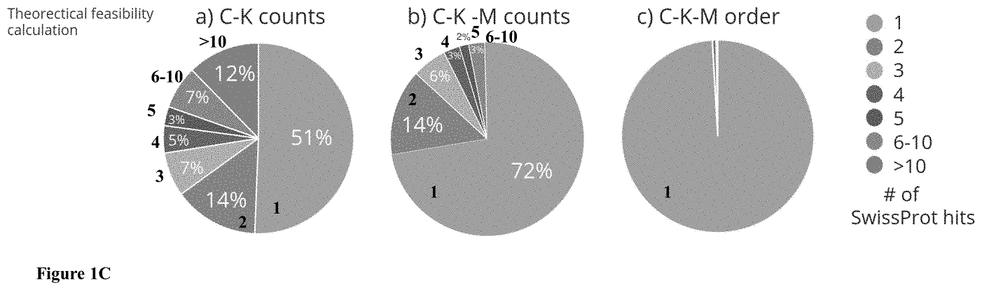

The theoretical identification values were calculated using the human proteome Swiss-Prot database, which contains 20,328 entries. For each entry we extracted the number of the target amino acids (C, K and M), as well as their order of appearance. For example, the p53 protein would either be characterized by its C, K, M counts (10, 20, 12, respectively) or by the sequence below: MKMMMKKCKMCKCMKMCCCMCCMMCC KKKKKKKMK (SEQ ID NO: 1), in which all intervening amino acids were deleted. Proteins having identical characteristic sequences (or C, K and M counts) are grouped together. A protein is identified when it is the sole member of a group. In the case of p53, both the C, K and M counts and the characteristic sequence gave a unique identification. The pie charts (FIG. 1C) distribute the proteins according to the size of the group in which they belong.

### A Protein Labeler Program

Each protein primary sequence was transformed into a string (B(i)) to which was assigned a value of 1, 2 or 3 corresponding to each of the three aa tags (K, C, and M), respectively; and 0 for all other aa in the protein sequence. To account for partial or nonspecific labelling a set of randomly selected labeled positions in the string were omitted according to a given labeling efficiency (η_{L}), and a set of artificial labeled positions were inserted according to a given nonspecific labeling efficiency (η_{NS}). It is important to note that nonspecific labeling did not affect all aa equally. For instance, in generating a barcode for lysine (K) positions, nonspecific labeling could only be inserted at positions of either threonine, serine or tyrosine (amino acids which have been shown to compete with NHS-ester-based labeling) with a probability of typically 1%. The strings were generated for the entire Swiss-Prot data base and were re-generated each time to simulate an uneven labelling of the same protein data sets, as well as whenever different values of η_{L} and η_{NS} were used.

### Finite Difference Time Domain Calculation of Plasmonic Fields

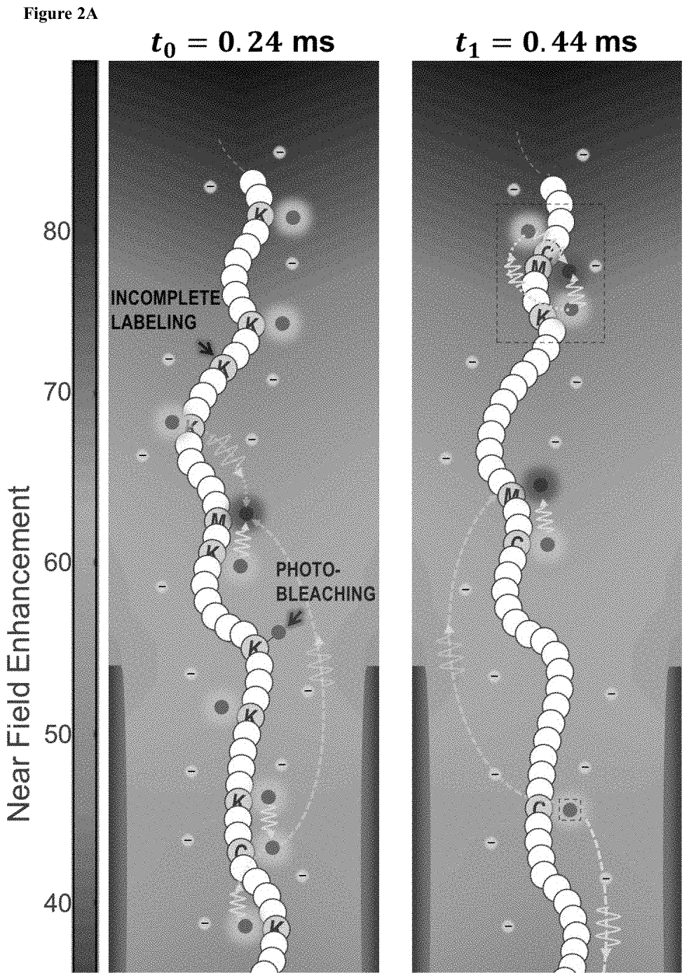

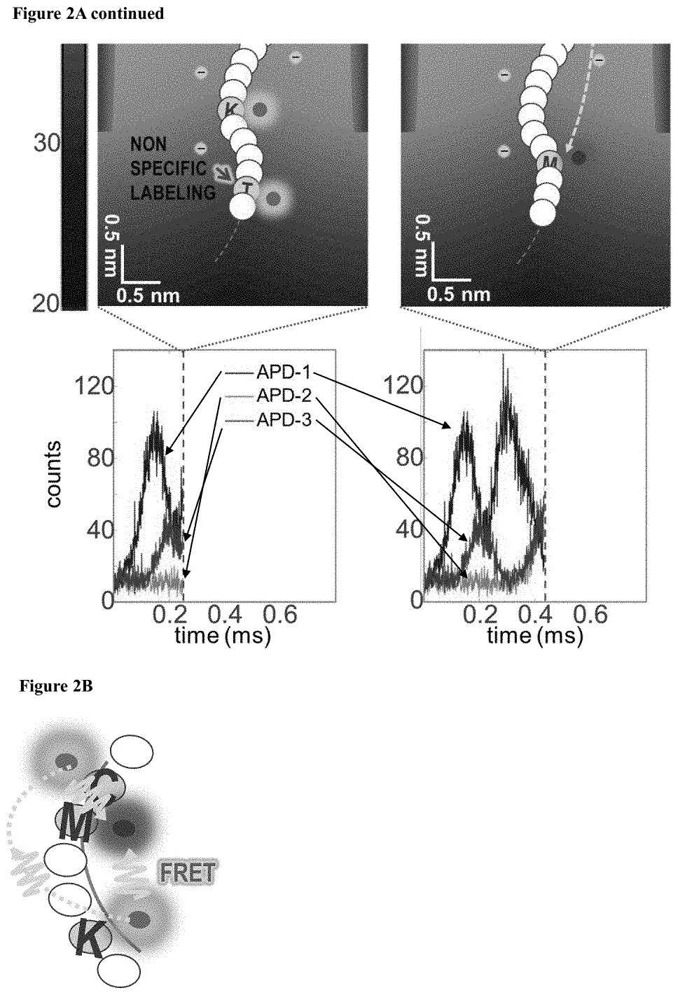

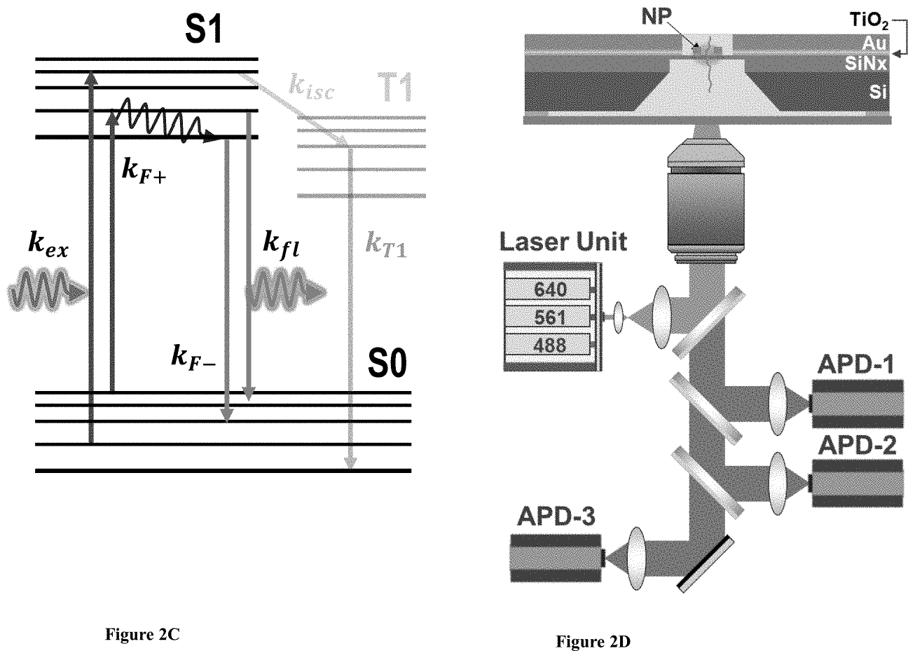

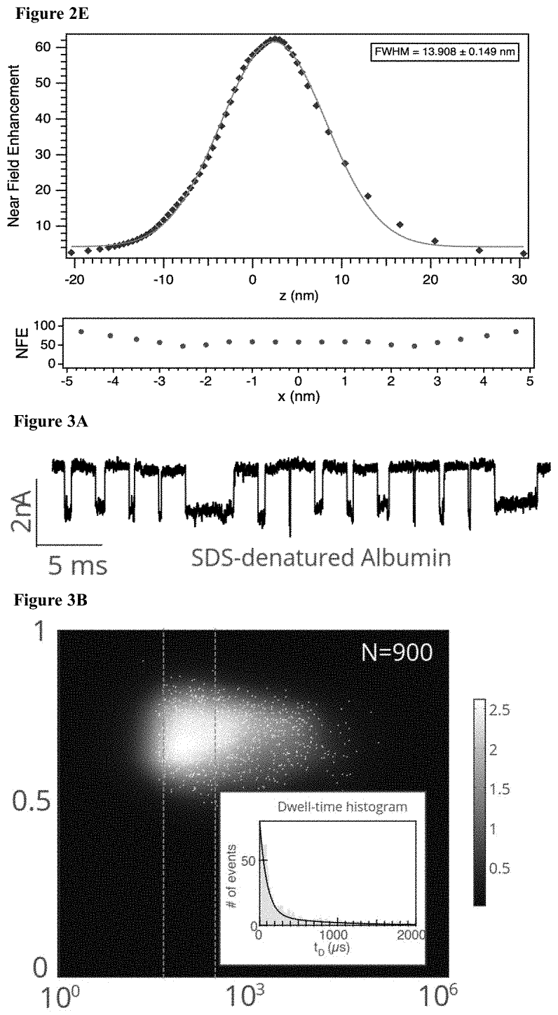

The three-dimensional near field enhancement of the plasmonic structure (2D vertical cross-section shown in FIG. 2A) was determined using a finite difference time domain (FDTD) method solving for Maxwell's time-dependent electromagnetic equations. The architecture over which the FDTD computations were performed comprised a 10 nm-tick silicon (Si) membrane—exhibiting a 3 nm-wide nanopore—on top of which a gold (Au) plasmonic structure was deposited (FIG. 2D). An additional 2 nm-thick titanium oxide (TiO₂) layer was inserted in between the Au structures and underlying Si membrane. The plasmonic structure consisted of a gold ring (inner and outer diameter of 12 and 32 nm, respectively, and a height of 40 nm) centered at the nanopore and embedded inside a gold nanowell (diameter of 120 nm and a height of 100 nm). Water was used as the immersion media.

The excitation field was modeled as a total-field scattering-field source (TFSFS) and the spatial sampling frequency was set to 5 nm⁻¹ (taking 60 frequency points over the 500-800 nm wavelength range). The FDTD boundary conditions consisted of 8-layer PMLs (perfectly matched layers) symmetric in the x axis and antisymmetric in the y axis thus minimizing the reflections and the computational cost, respectively. Frequency domain power monitors only were incorporated in the simulation to determine the near field enhancement in the vicinity of the nanopore. All numerical simulations were performed using Lumerical FDTD Solutions (Lumerical, Inc).

### Simulation of Nanopore-Based Optical Sensing of Proteins

To simulate the translocation of the linearized protein through the nanopore, there was assumed a unidirectional motion with steps of a single aa length (Δ≈0.35 nm) and an average velocity u (cm/s). To account for thermal fluctuations in this process, a random noise term δu was added at each step (δu can be positive or negative). Hence the simulation step time of the i-th aa was defined as τᵢ=Δ/(u+δu). The average protein velocity value was typically ˜0.2 cm/s, based on experiments using SDS denatured proteins in solid-state nanopores as shown in FIG. 3. Additionally, faster translocations (2 cm/s) was tested. The fluorescence emission rate of each fluorophore n in the system K_{fl,j,n}(t) was modeled as a two-state system:

K_{fl,j,n}(t)=k_{fl,j}P_{j,n}(t) Eq. 1

where j=1 . . . 3 correspond to each of the three excitation/emission channels, k_{fl} the fluorescence transition rate and Pₙ(t) the occupation probability of the excited molecular state S1. The fluorophores are excited by up to three laser lines corresponding to the three channels, that form sub-wavelength excitation volumes by means of a plasmonic nanostructure or total internal reflection. The axial full width at half maximum of our Gaussian excitation volume Iₑₓ is defined as ξ and is allowed to vary from 5 nm to 200 nm in order to account for broad possible experimental conditions. The emitted light from the three-color channels is assumed to be acquired with given efficiencies ηⱼ, which include both the optical transmission efficiencies and the photodetector efficiencies. The photon counts Iᵢ^{j} at each channel j during each step i of the protein translocation is then determined by summing the emissions of all the fluorophores n that resides within the excitation volume. Namely:

$${I}_{i}^{j} = {η}_{j} {\sum }_{n} {K}_{f l , j , n} ( {t}_{i} ) + {k}_{b g} {τ}_{i} = {η}_{j} {\sum }_{n} {k}_{fl , j} {P}_{j , n} ( {t}_{i} ) + {k}_{bg} {T}_{i} \\ \{ \begin{matrix}{P}_{j , n} ( {t}_{i} ) = {P}_{j , n} ( {t}_{i - 1} ) + ( \frac{{k}_{e x , j} ( n )}{{k}_{j} ( n )} - {P}_{j , n} ( {t}_{i - 1} ) ) ( 1 - {e}^{- {k}_{j} ( n ) {t}_{i}} ) & Eq . 3 \\ {k}_{j} ( n ) = {k}_{e x , j} ( n ) + {k}_{S 1 , j} = {σ}_{e x , j} \frac{{I}_{e x , j} ( n ) {λ}_{e x , j}}{h {c}_{0}} + {τ}_{S 1 , j}^{- 1} & Eq . 4\end{matrix} \qquad Eq . 2$$

where k_{bg} is the background emission rate, tᵢ the time at which step the translocation occurred such that tᵢ−tᵢ₋₁=τᵢ, k_{ex,j}(n) is the excitation rate of the fluorophore n of channel j, σ_{ex,j} is its absorption coefficient, λ_{ex,j} is the excitation wavelength and τ_{SI,j} is its excited state lifetime.

The number of cycles (S0→S1→S0) undergone by each fluorophore was capped to account for photobleaching according to a decaying exponential distribution. Specifically, the maximum number of cycles performed by each fluorophore before photobleaching was given by a random number drawn from a decaying exponential distribution with a characteristic decay of ˜10⁶. Finally, we applied a Poisson distribution to the photon counts Iᵢ^{j} to simulate shot noise.

To include energy transfer (such as Førster Energy Transfer and homo-transfer) in this system a 2D distance matrix was calculated for each fluorophore in the system. The distances between the labelled aa's (or fluorophores) in each linearized protein were subsequently used to calculate the Forster energy transfers of each fluorophore from and to each of its neighboring emitters. As a proxy for the exact energy transfer, two additional transition rates accounting for energy gain and loss were incorporated in the fluorophore two-state model:

$$\{ \begin{matrix}{k}_{FRET + , j} ( n ) = \frac{1}{h {c}_{0}} {\sum }_{i} {\sum }_{m \neq n} {σ}_{ex , i} {I}_{ex , i} ( m ) {E}_{n ← m} {λ}_{ex , i} & E q . 5 \\ {k}_{FRET - , j} ( n ) = \frac{{σ}_{ex , j} {I}_{ex , j} ( n ) {λ}_{ex , j}}{h {c}_{0}} {\sum }_{i} {\sum }_{m \neq n} {E}_{m ← n} & Eq . 6\end{matrix}$$

where E_{m←n}=(1+(|xₙ−xₙ|/R_{0,m←n})⁶)⁻¹ is the FRET energy transfer efficiency from fluorophore n to m, xₙ is the position of fluorophore n along the denatured protein and R_{0,m←n} is the Forster-radius of the (n, m) dye pair when considering an energy transfer from fluorophore n to m. The transition rates k_{ex,j}(n) and kⱼ(n) in Eq. 4 were corrected to account for FRET accordingly:

$$\{ \begin{matrix}{k}_{e x , j} ( n ) \to {k}_{e x , j} ( n ) + {k}_{F R E T + , j} ( n ) \\ {k}_{j} ( n ) \to {k}_{j} ( n ) + {k}_{F R E T + , j} ( n ) + {k}_{F R E T - , j} ( n )\end{matrix}$$

The code was implemented using MATLAB, and the optical readouts of the three channels were determined by running this procedure for each labeling string.

### Protein Classification and Mapping of Optical Reads to Protein IDs

For the purpose of a multi-class (the human proteome comprises more than twenty thousand proteins) classification of time-series that exhibit specific patterns, convolutional neural networks (CNN) were used that have shown great promise in the field of pattern recognition, including image classification, which similarly requires tens of thousands of classes. Specifically, the python deep learning package Keras was used on a four GPU architecture (NVIDIA Tesla K40), which leads to a CNN whole-proteome training time of −2 h only. The CNN model relied on four sequential layers—a convolutional layer, a normalization layer in which dropout was applied and a pooling layer—followed by a multi-layer perceptron. In brief, the convolutional layer filters (at a given step or stride size) the translocation time-series with a large set of kernels of a specific size. The resulting activation or feature map it provides is further transformed by the normalization layer such as the mean and standard deviation of the activation map approach zero and one, respectively. Next, the dropout circumvents overfitting of the CNN to the training dataset by setting a random subset of activations to zero. The last pooling layer performs a down-sampling operation on the activation map to further prevent overfitting of the training dataset and the computational load. The multi-layer perceptron consists of a single densely connected neural network layer, each neuron outputting the probability of belonging to the class it represents (‘softmax’ activation function).

The hyper-parameters were optimized according to standard procedures, that is maximizing the accuracy of the CNN trained over five to ten epochs per hyper-parameter set. Once finely adjusted, the CNN was trained using twenty epochs to yield the greatest accuracy. The protein identification accuracy as determined by the CNN was calculated as the fraction of correctly classified translocation events from the test dataset. The dataset was randomly partitioned into five pairs of training and testing sub-sets, and for which the identification accuracy was determined. The final accuracy was calculated as the average between them where a typical test set included ˜400,000 translocation events.

### SDS-Denatured Protein Translocation Experiments

Solid-state nanopores were fabricated using a laser drilling method in 17 nm-thick SiNₓ membranes as is known in the art. Human serum albumin (Biological Industries Inc. 30-O595-A) was first treated by TCEP (5 mM) at room temperature for 30 min to break disulfide bonds and subsequently denatured at 90° C. for 5 min in PBS with 2% sodium-dodecyl sulfate (SDS). The resulting albumin concentration was further diluted (100:1) to <1 nM in buffer (PBS/0.4M NaCl/0.1% SDS/1 mM EDTA) for nanopore translocation experiments performed under a 300 mW bias. A custom-made LabVIEW interface was used to acquire and analyze each event. Scatter plots and dwell-time distributions were generated using Igor Pro (Wavemetrics).

### Example 1: Simulation of Nanopore-Based Recognition of Proteins

In the method of the invention, proteins extracted from any source (serum, tissue or cells), are denatured using urea and SDS (FIG. 1A). Three amino-acids lysine (K), cysteine (C) and methionine (M) are labeled with three different fluorophores using three orthogonal chemistries: the primary-amines in lysines are targeted with NHS esters; thiols in cysteines are targeted with maleimide groups, and methionines are labeled using the two-step redox-activated chemical tagging. The negatively charged SDS-denatured polypeptides are electrophoretically threaded, one at the time, through a sub-5 nanometer pore fabricated in a thin insulating membrane to ensure single file threading of the SDS-coated polypeptide. The voltage, nanopore diameter and other factors, such as solution viscosity are used to regulate the protein translocations speed. The nanopore is illuminated using laser beams for multi-color excitation. The excitation volume (FIG. 1A, yellow highlighted region) is centered with the nanopore, and importantly, its axial depth is confined by plasmonic focusing of the incident electromagnetic field. Consequently, depending on the excitation depth, either a single, or multiple, labeled amino acids will be simultaneously illuminated, during the passage of the protein. Three-color fluorescence time traces (“fingerprints”) are recorded for each protein passage and are classified using deep-learning (FIG. 1B).

The theoretical likelihood of protein ID can be tested by calculating the percentages of unique matches of all proteins in the human Swiss-Prot database based on the number and the order of appearance of three amino-acids only. Simply counting the number of K, C and M residues in each protein identifies 72% of the total proteins uniquely, and another 14% identified as either one of two proteins in which one of them is the correct match (See Materials and Methods). Moreover, the percentage of uniquely identified proteins is close to 99% with the determination of the KCM order of appearance along all proteins in the human proteome database (FIG. 1C). Thus, in principle, the boundaries for the expected ID accuracies fundamentally permit whole-proteome, single-protein, identification.

The theoretical analysis shown in FIG. 1C may be considered as an upper limit for the accuracy of a protein ID method based on the three amino-acid labelling. However, it ignores experimental limitations, such as the sensing spatial and temporal constraints, the labelling efficiency and the photophysical properties of fluorophores. These factors are likely to impact the accuracy of the protein ID method, and hence must be considered. To this end there was developed a detailed photophysical model to numerically calculate the time-dependent photon emission during the passage of each SDS-denatured protein through a solid-state nanopore. The model consists of three layers: first, Finite Difference Time Domain (FDTD) computations were used to evaluate the expected electromagnetic field distribution for a simple plasmonic structure fabricated on top of the nanopore (Materials and Methods). Second, an amino-acid labelling simulation was applied to each protein, in order to generate partial labelling of each of the three target amino-acids. Finally, SDS-denatured proteins were allowed to slide through the plasmonic nanopore complex while illuminated at three distinct wavelengths. The expected detected photon emissions were calculated at each step of the protein translocation taking into account the photophysical properties of the fluorophores, as well as energy transfer (FRET), bleaching kinetics and collection efficiencies. This allowed the generation of detailed photon emission time traces for each and every protein translocation.

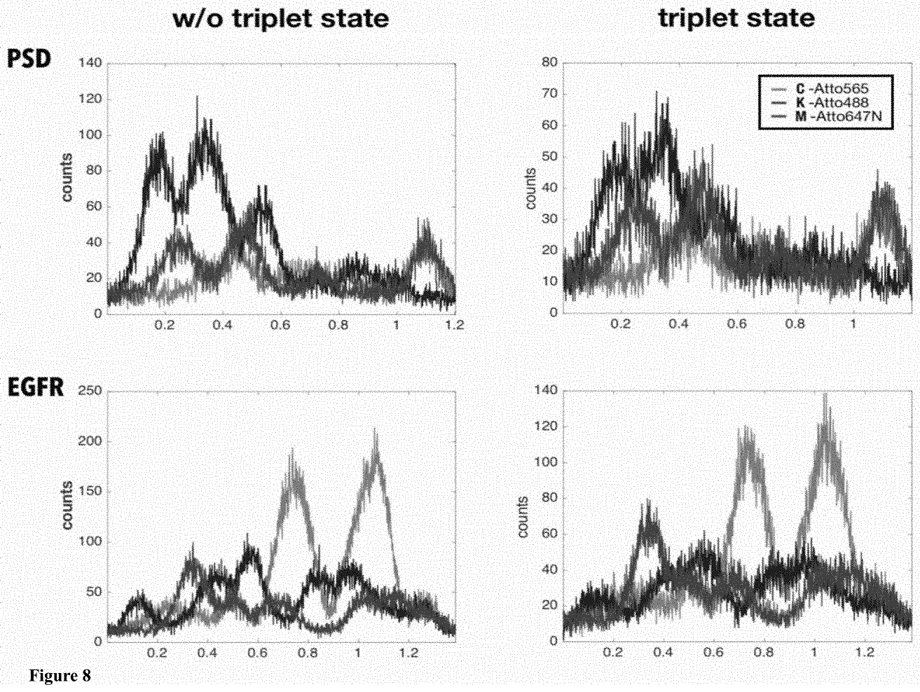

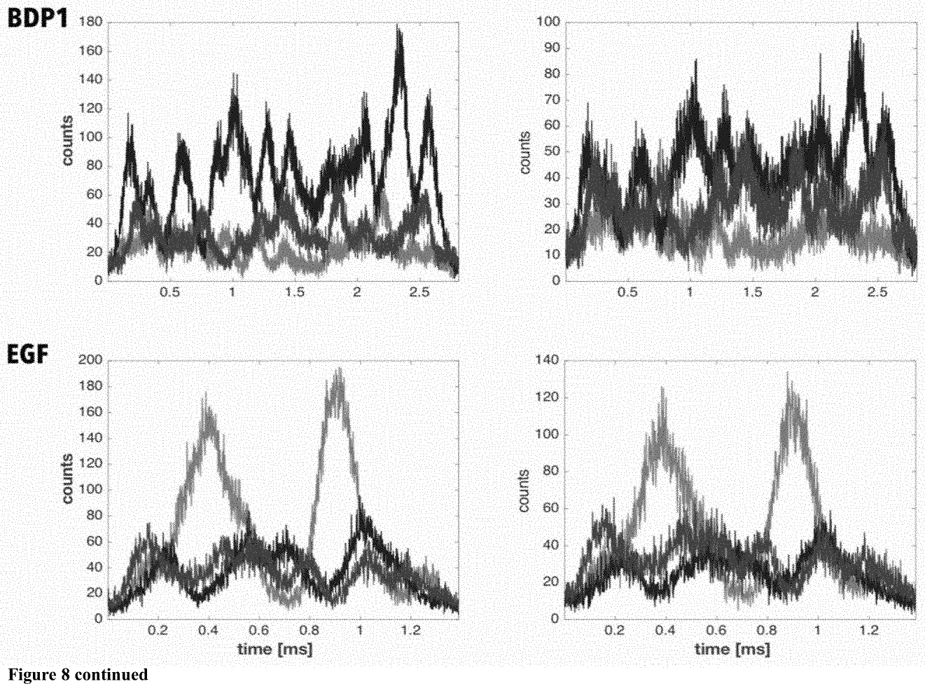

To illustrate this method, FIG. 2A schematically shows snapshots of the system at two time points during the passage of the PSD protein. This figure is plotted in scale to illustrate the relative dimensions of the plasmonic field, the nanopore and the SDS-coated polypeptide chain (marked as orange layer around the chain). Specifically, the axial FWHM of the plasmonic field is 20 nm calculated from the FDTD field distribution, and the nanopore diameter is 3 nm. Each protein was modeled as a fully-denatured, SDS-coated, wormlike polymer, translocating across the nanopore at an instantaneous velocity uᵢ= [symbol] u [symbol] +δuᵢ where [symbol] u [symbol] is its average velocity, and the random term δuᵢ accounts for thermal fluctuations in its motion. Since the SDS-coated biopolymers have a Kuhn length of approximately 7 nm, they can be assumed to be partially-stretched (unfolded) wormlike polymers during translocation through a sub ˜5 nm pore. Moreover, when threaded through a 3 nm pore, the roughly 2 nm wide SDS-coated proteins are confined laterally in a small volume in the nanopore proximity where the electromagnetic field remains nearly constant. Hence, in this study the protein translocations can be treated as one dimensional. The excitation profile calculated from the FDTD simulations was approximated by a one-dimensional Gaussian function as shown in FIG. 2E. The fluorescence emission rate of each labeled amino-acid while passing through the excitation zone was modeled as a two-state system (FIG. 2C), as described in the Materials and Methods section. Triplet state transition rates, which may result in microsecond-long dark-states were also considered based on literature values of three specific fluorophores (FIG. 8). Energy transfer rates were explicitly taken into consideration (FIGS. 2B and 2C), which directly depend on the amino-acid sequence, as well as photo-bleaching rates (indicated by dotted yellow lines and solid grey arrows respectively throughout FIG. 2). At each time step of the simulation the emitted light from all fluorophores residing in the excitation zone were split to three spectrally-resolved, photon-counter channels as shown in FIG. 2D. In addition to the collection and detection efficiency of each channel, photon statistics were also considered by incorporating shot-noise.

The labeling efficiency was modeled by randomly positioning fluorophores at the K, C and M amino-acid, such that in each protein only a fraction Γᵢ of them (j represents K, C or M) was actually labelled (indicated by purple arrows in FIG. 2A). In all the following computational results presented the three amino-acids, K, C and M were labelled by Atto488, Atto565 and Atto647N fluorophores, and the fluorophores properties were taken into account when simulating the photon emission rates. Additionally, we introduced cross-labelling efficiency (green arrows in FIG. 2A), although this is known to be negligible.

In order to estimate the translocation velocity of SDS-denatured polypeptides electrical translocation measurements using SDS-denatured albumin (585 amino-acids) proteins were performed using ˜4 nm-wide solid-state nanopores, as described in the Materials and Methods section. Representative translocation events measured at a bias voltage of V=300 mV, in which a single blockage current level is observed, are shown in FIG. 3A. Examining a statistical set of >900 translocation events showed a single blockade current level (I_{B}=0.7) indicative of single-file polypeptide translocations. This experiment supports the assumption that proteins are likely to be fully denatured as they thread through the narrow nanopore, in agreement with what is known in the art. FIG. 3B displays an overlay of the scatter plot of the fractional blockade current I_{B} versus the translocation dell-time t_{D}, with its corresponding density map. The area delimited by the dashed red curve approximates the typical full-width-half-maximum of a Gaussian centered on the characteristic dwell time (94.3±7.2 us as determined by the histogram shown in the inlet panel). Accordingly, the mean translocation velocity is estimated to be 0.2 cm/s. Notably, this velocity is slower than a previous report, presumably due to the fact that in this experiment a much smaller nanopore was used.

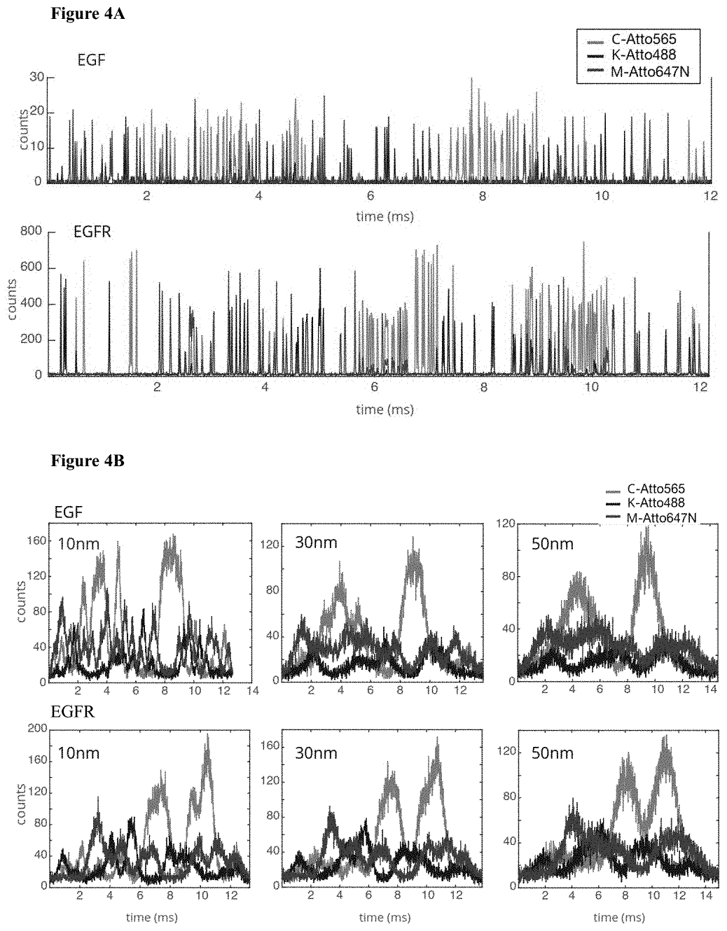

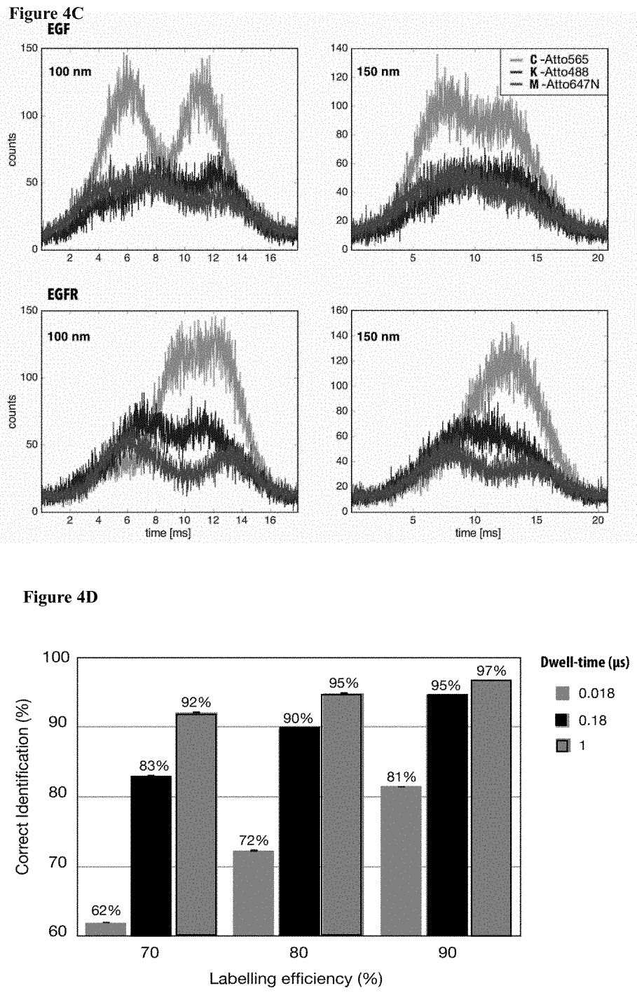

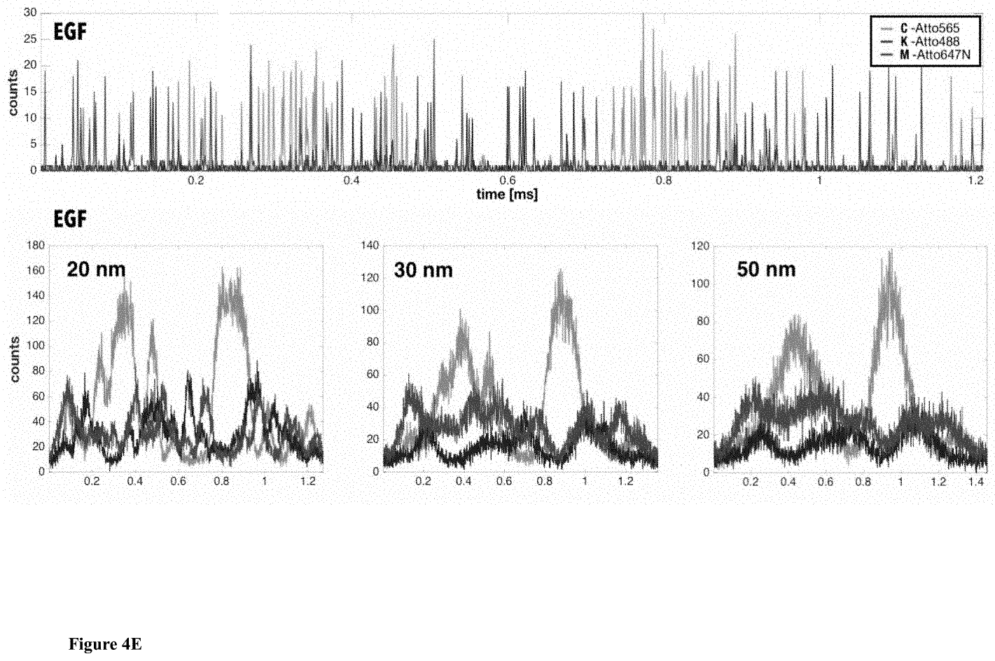

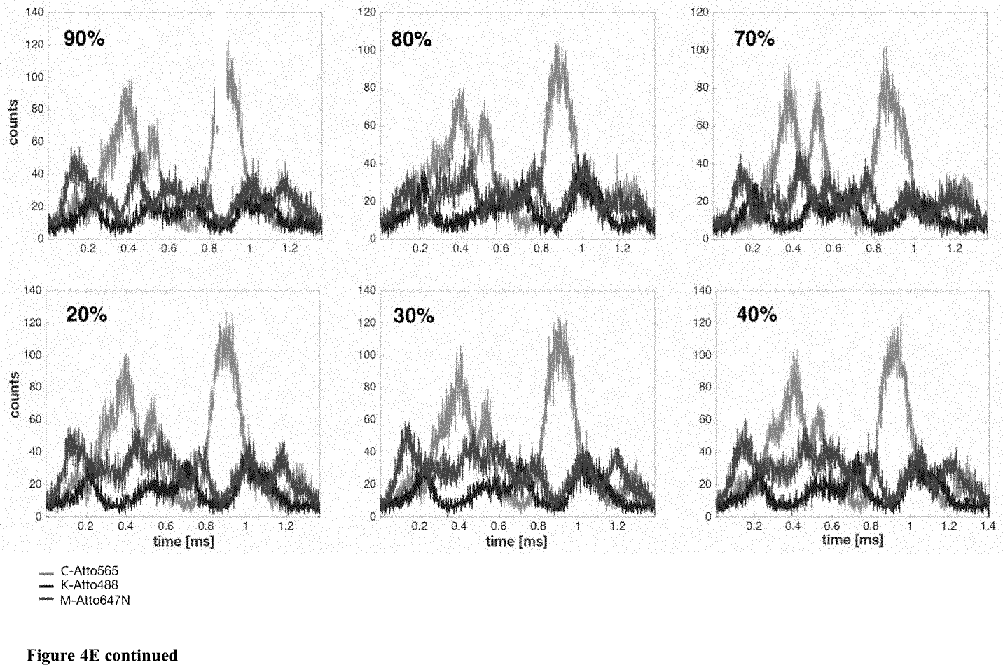

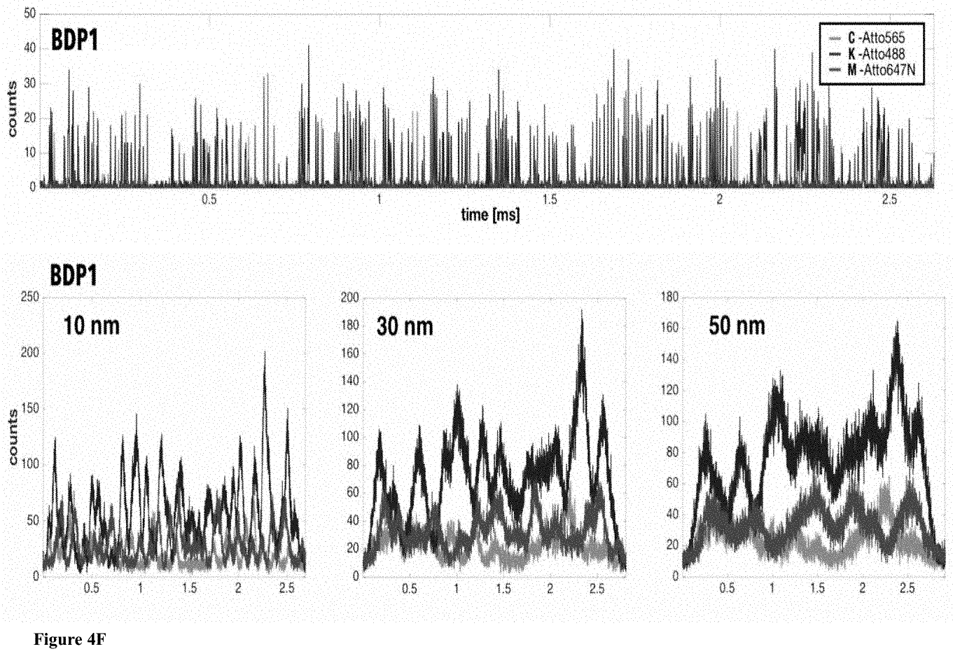

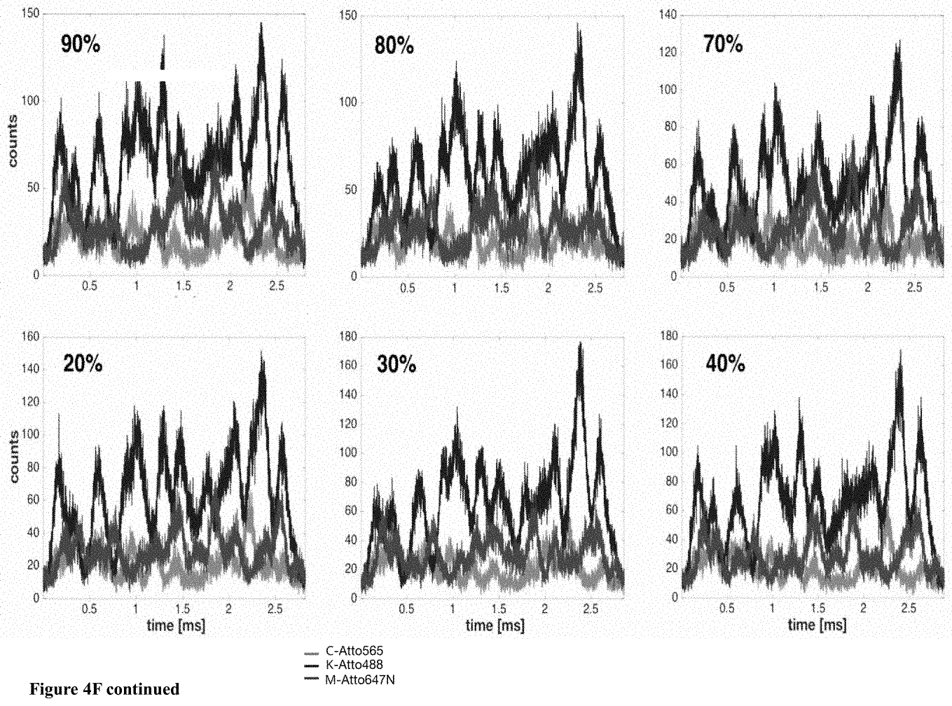

Initial focus is placed on simulated optical signals calculated for two proteins having nearly the same length: the EGF precursor, and its receptor EGFR (1208 and 1210 amino acids, respectively). Under near-ideal experimental conditions (100% labelling, 0.5 nm resolution, and velocity of 0.035 cm/s) their tri-color fingerprints were readily distinguishable from each other, despite similar K, C and M compositions, and followed the actual K,C,M amino acid order in each protein (FIG. 4A). Next, the protein translocation simulations were extended under much lower spatial resolutions, lower labelling efficiencies and higher translocation velocities. As expected, in the more realistic conditions individual fluorophore photon bursts, associated to single K, C or M residues, can no longer be resolved. Instead, the resulting signals appear as continuous tri-color fingerprints of each protein translocation. Importantly, however, the fingerprints, even at the poorest resolution of 50 nm maintain an overall pattern characteristic of each protein (FIG. 4B). Analyzing >5·10⁷ single protein translocations events, under different conditions suggest that even at 100 nm resolution some characteristic features of each protein are preserved (FIG. 4C). Moreover, it is expected that small variations in the nanopore size would result in different translocation velocities. To evaluate this effect, the translocation simulation experiments were repeated at mean values of 0.035, 0.2 and 2 cm/s and increasing the translocation velocity fluctuations (20%, 30% and 40% of the mean velocity). The results (FIG. 4D-F) suggest that as long as the velocity is in the order of ˜0.2 cm/s (or below) in accordance with the experimental result (FIG. 3), the identification accuracy remains sufficiently high.

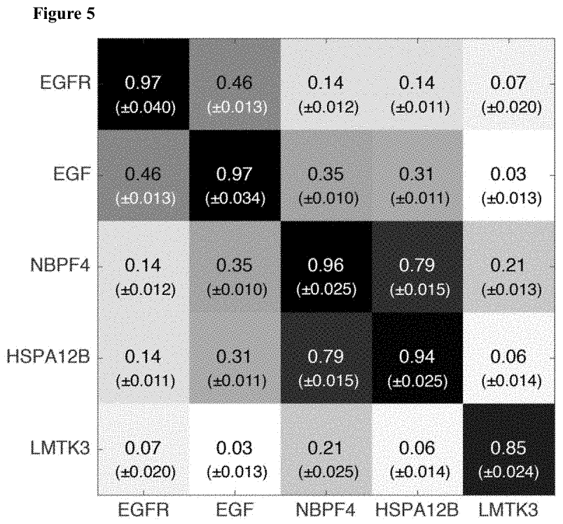

The similarity among repeated translocations of the same proteins, which were subject to different labeling and random velocity fluctuations, was tested by evaluating the Pearson correlation coefficients between all pairs of 50 translocation repeats of the same protein. The results, showed in all cases high values (0.85-0.97) when considering autocorrelation (FIG. 5, diagonal values). In contrast, attempting to cross-correlate among 5 different, randomly chosen, proteins produced in most cases much lower Pearson coefficient values (0.03-0.35). Obviously, this is just a small fraction of all possible cross-correlations. However, even as is, this sample of data suggests that the protein translocation simulator generates highly reproducible signals.

### Example 2: Whole-Proteome Protein ID Using Deep-Learning Classification

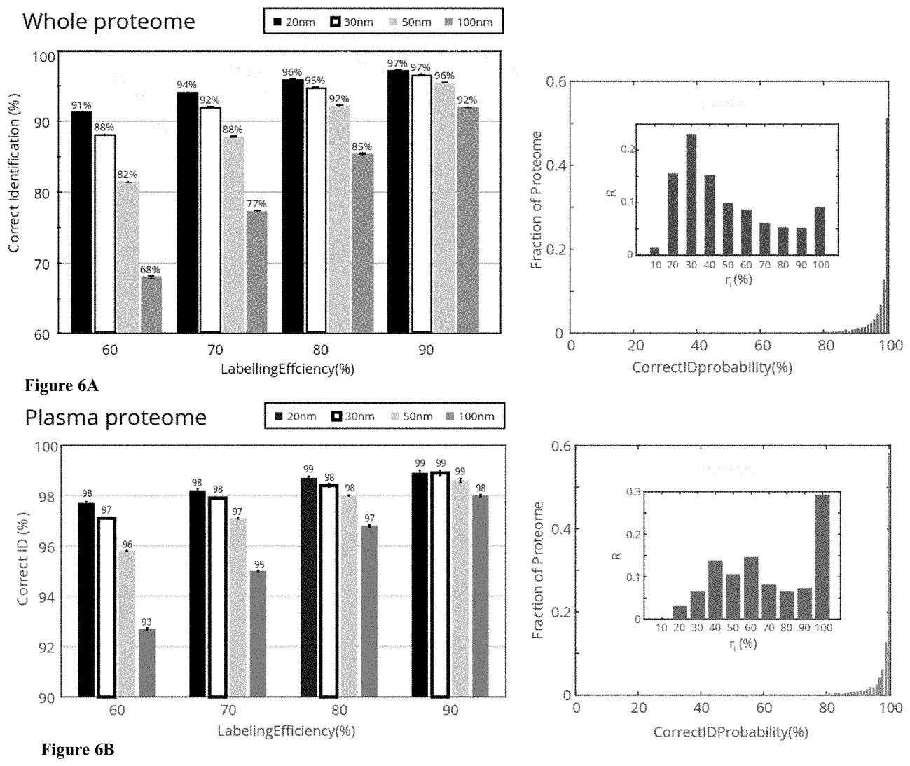

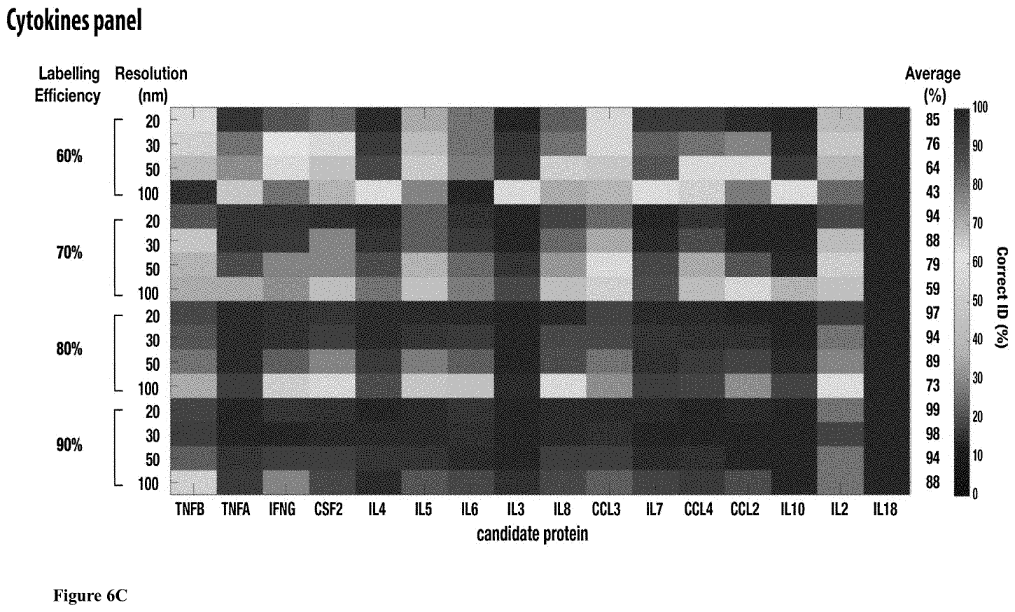

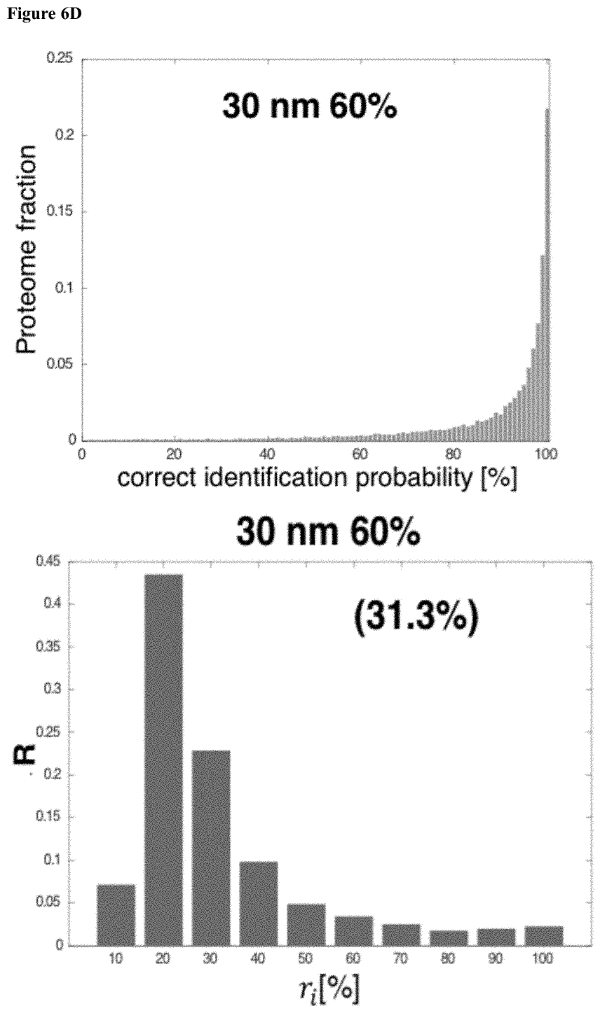

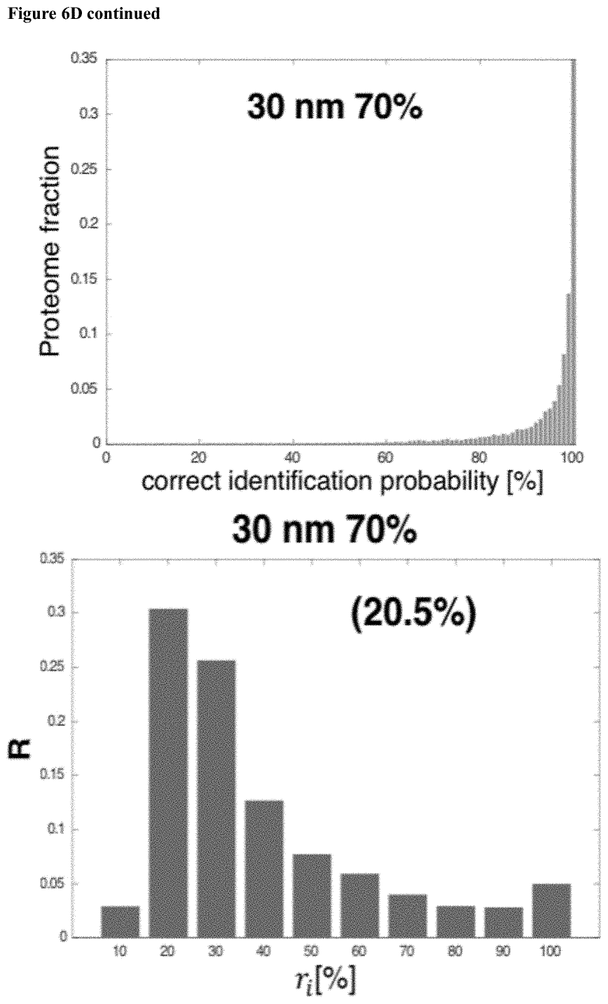

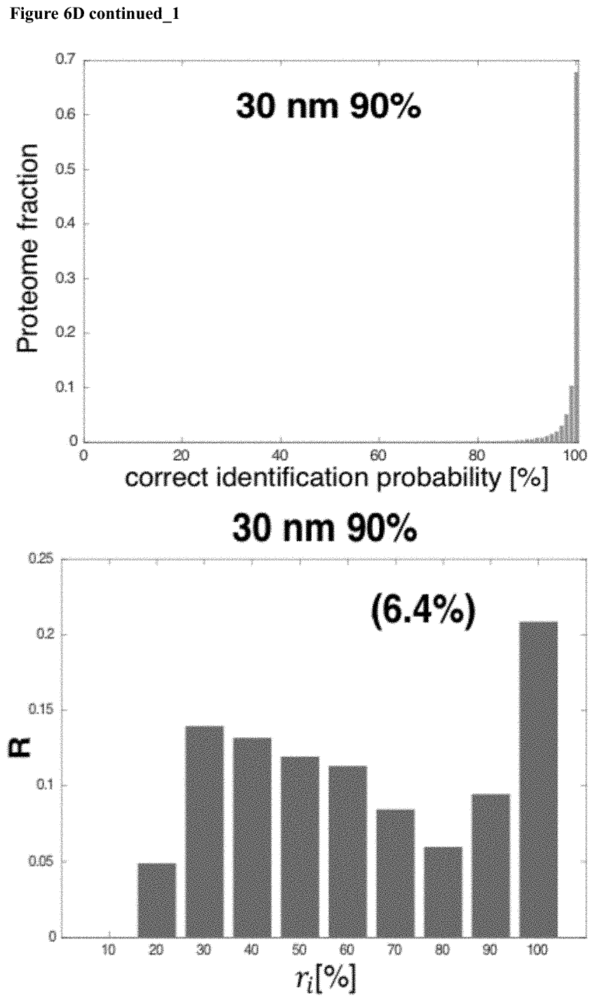

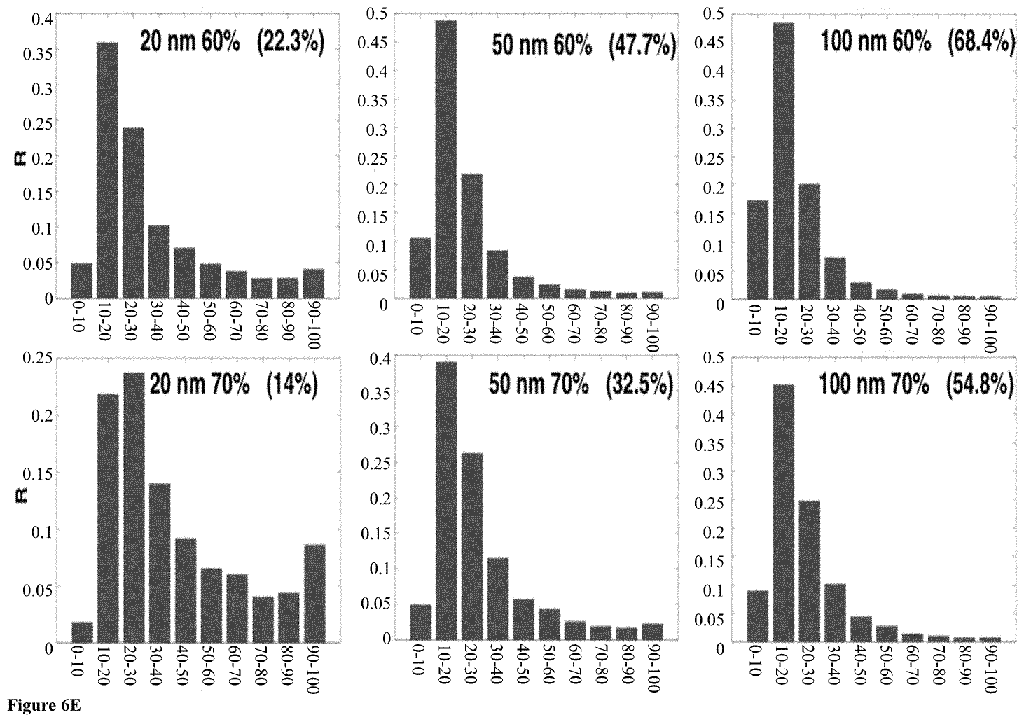

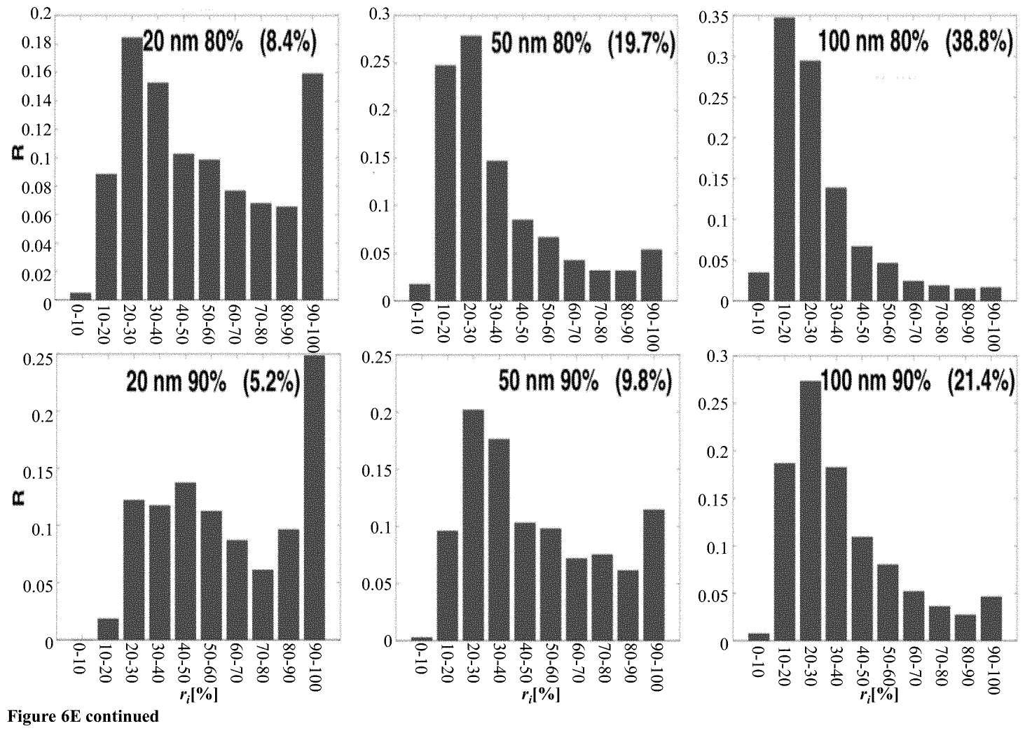

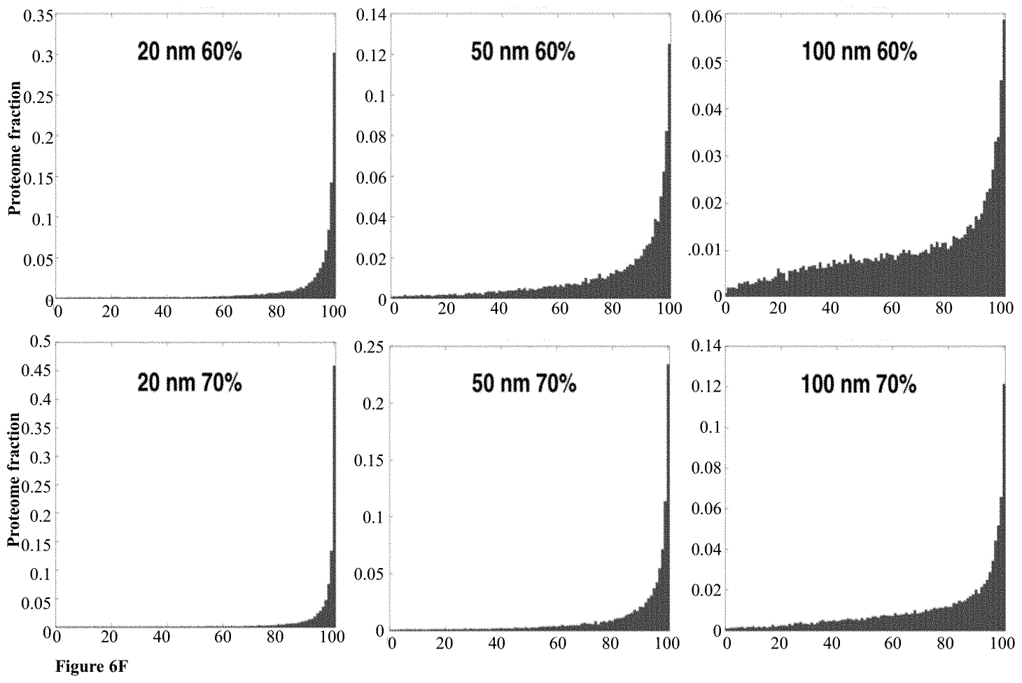

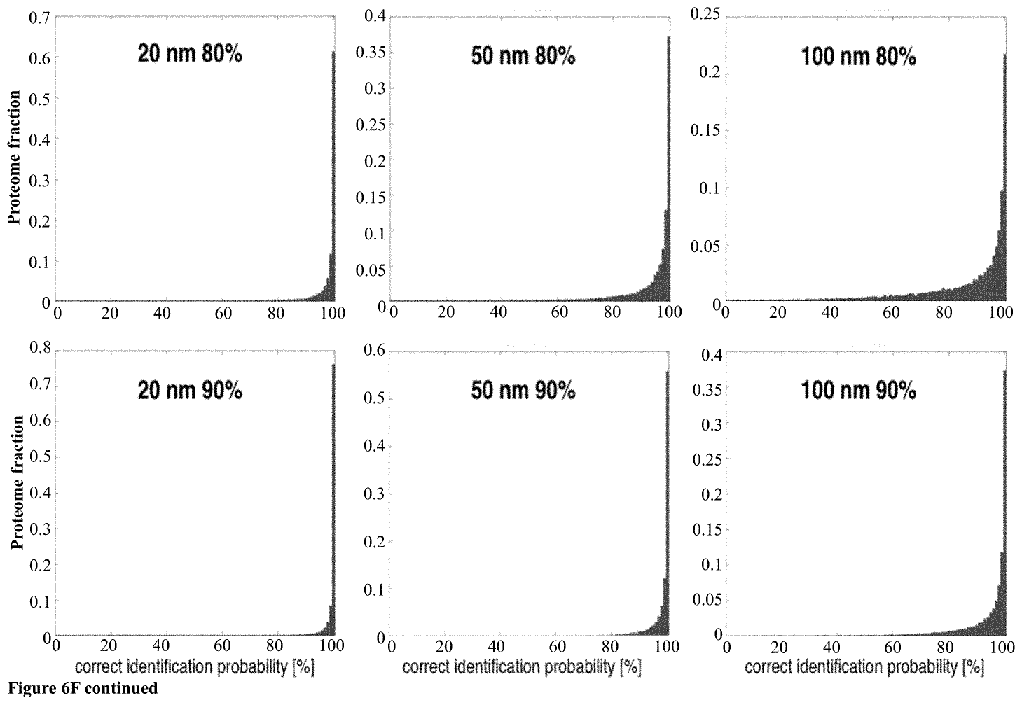

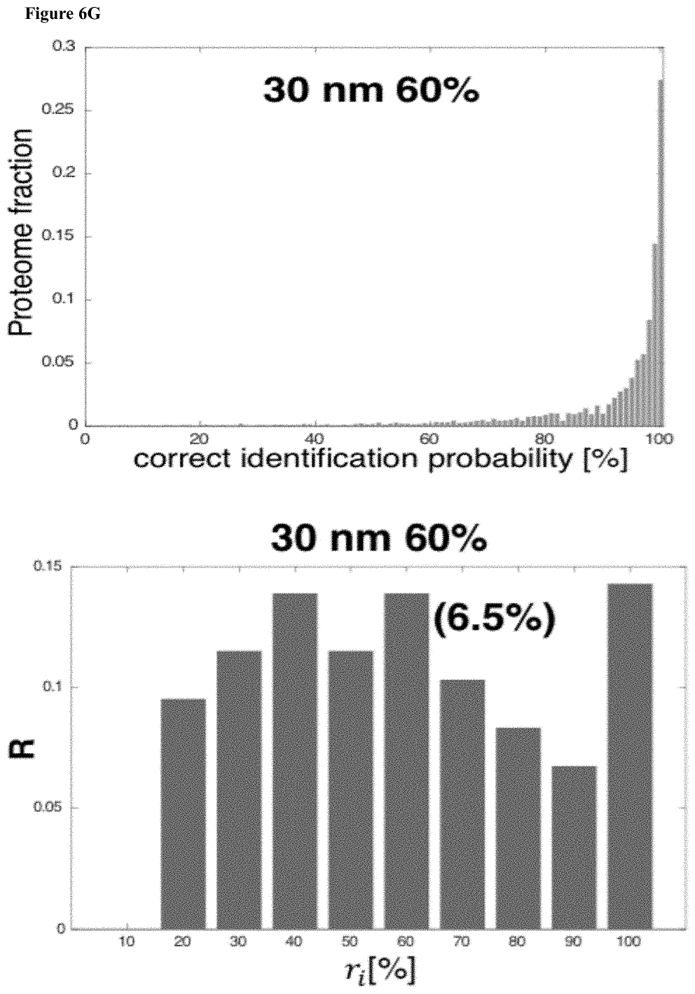

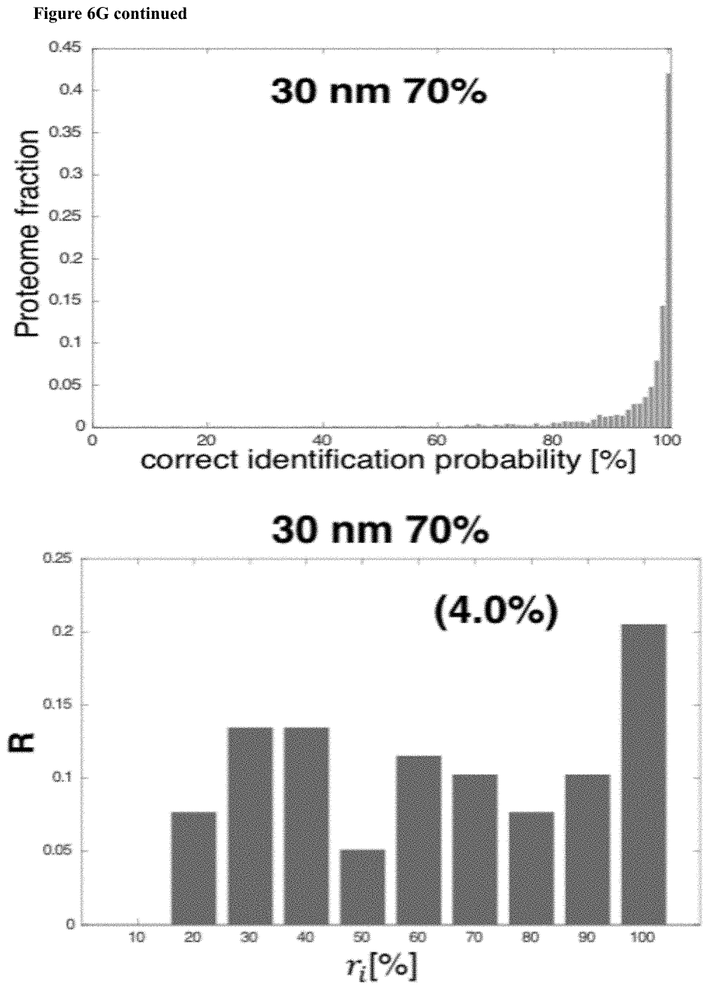

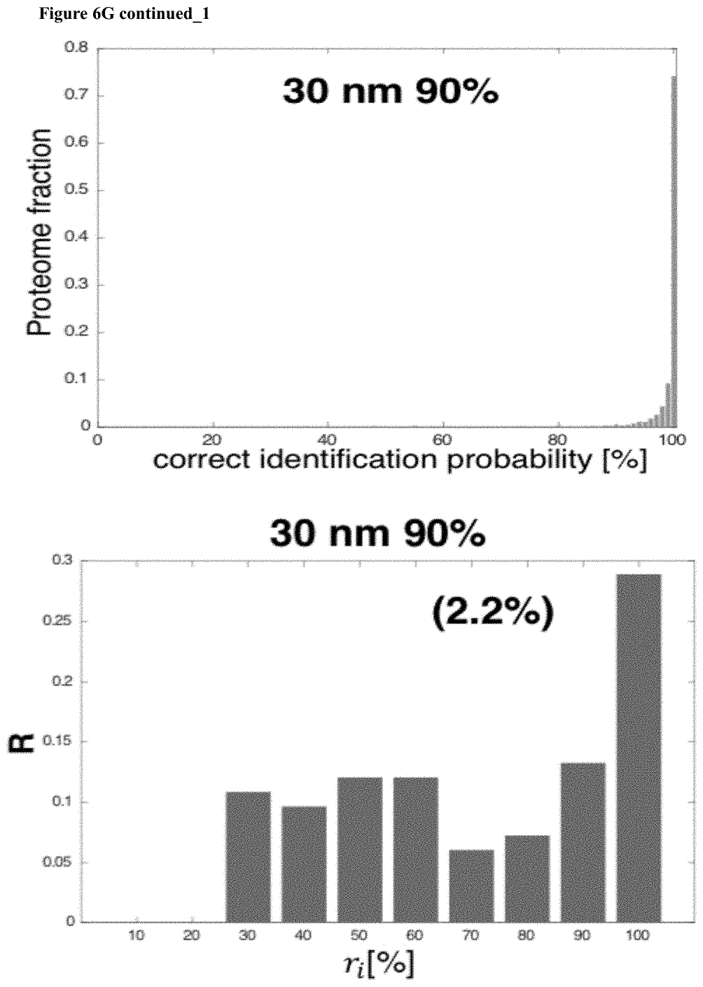

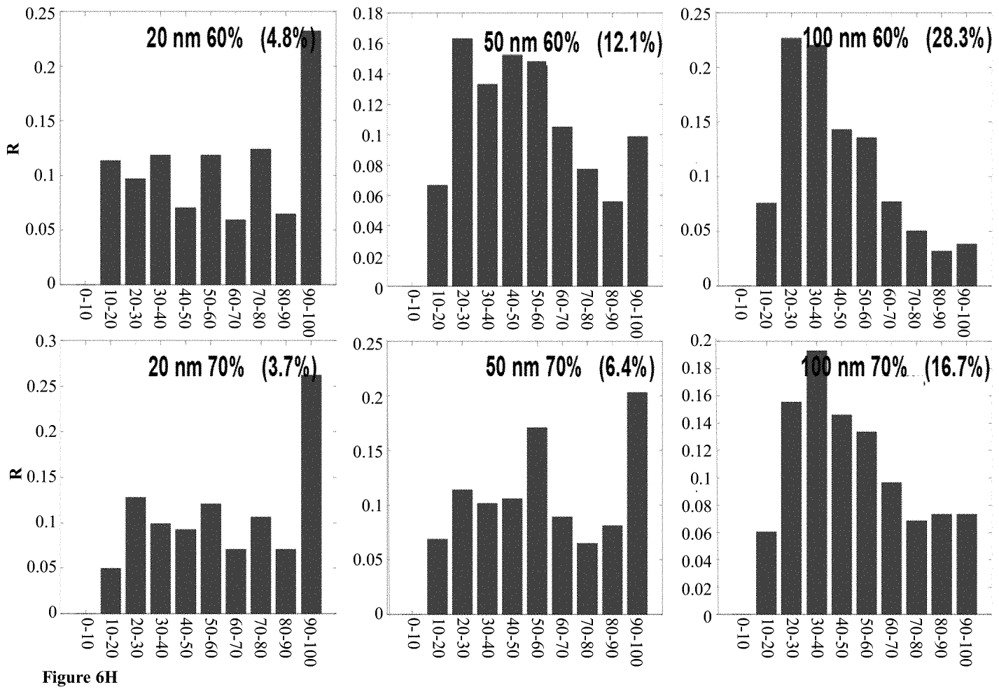

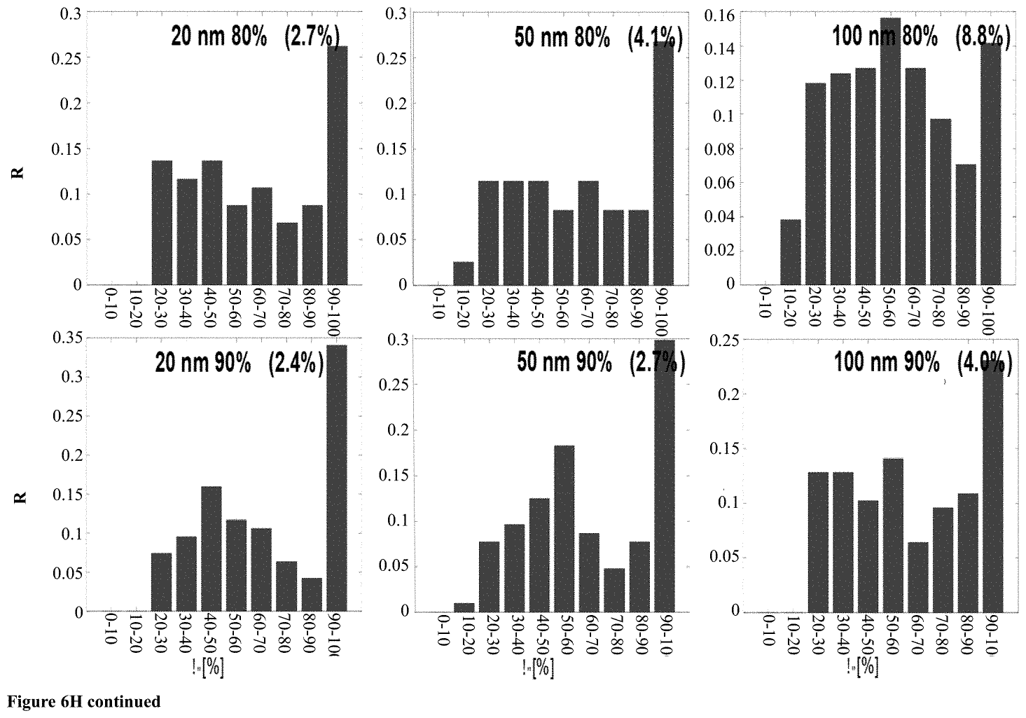

Next the simulations were vastly scaled-up to include thousands of different proteins, each one repeated hundreds of times under different labeling efficiencies, translocation velocities and spatial resolutions. The accurate classification of noisy, low-resolution, time-dependent signals is often encountered in areas such as image and speech recognition and is effectively handled by Convolutional Neural Networks (CNN) approaches. It was postulated that, provided sufficient training, the CNN approach would be able to identify most proteins based on the tri-color fingerprints. To check this hypothesis, deep-learning whole-proteome analyses were set up. First, the CNN network was trained using a large dataset containing at least 80 individual nanopore passages of each protein in the Swiss-Prot database. Then the CNN was presented with new protein translocation events and queried as to the protein identity. This procedure was repeated at least 5 times for whole-proteome analysis allowing the establishment of the mean ID accuracy and its standard deviation, for 16 different experimental conditions (FIG. 6A). Starting with the highest labelling efficiency (90%, right-hand set) it was observed that 96%-97% of all protein translocations were correctly identified, as long as the spatial resolution was <50 nm. The correctly identified protein fraction dropped down to 92% using a 100 nm resolution. A similar pattern can be observed for the other labelling efficiencies with somewhat lower numbers. In the worst-case scenario considered here (100 nm resolution and only 60% labeling efficiency) the CNN nevertheless was able to correctly classify 68% of all translocation events, similar to the ideal case considered in FIG. 1C (C, K, M counts only). In other words, despite the fact that 40% of the target amino acids were not labeled, and the resolution of the probing was about a third of the optical diffraction limit, the pattern recognition algorithm identified correctly nearly 70% of all protein translocation events. When the labelling efficiency was improved to the expected standards (between 70%-90%), and the sensing resolution assumed to be in the 20-30 nm, the correct identification of all translocation was roughly 95%. Increasing the translocation speed of proteins by nearly two orders of magnitude to 2 cm/s (an order of magnitude higher than the mean measured velocity in FIG. 3), reduced the ID accuracy (FIG. 4F). However, for high labeling efficiencies (80% and 90%) the ID accuracy was still high (72% and 81%, respectively).

In addition to the mean accuracies, the CNN algorithm produces a “confusion matrix”, which presents the number of times each and every protein x was identified as protein y (where x and y could be any of the proteins in the set). This information was used to calculate the probability density function (pdf) of correct ID for each and every classification set, namely the likelihood that a given protein is correctly identified with probability p. The pdf of correct ID calculated for the case of 30 nm resolution and 80% labelling efficiency (FIG. 6a, right panel) indicates that 51%, 71% and 89.2% of proteins were correctly identified with probability of 1.0, 0.98-1.0 and 0.9-1.0, respectively. The probability distributions for all other conditions are shown in FIGS. 6D-E.

The results for misclassified proteins were also analyzed. Specifically, it was of interest to know whether a misclassified protein is likely to be a specific protein, or randomly misclassified. To investigate the degree of randomness in misclassification, first were selected proteins that had at least 10% misclassified events. Then, was determined the fraction of identical mismatch rᵢ=maxᵢ nᵢⱼ/Nᵢ for each protein i, where nᵢⱼ is the number of translocation events misidentified to protein j and Nᵢ the total number of misclassified translocation events. With this a high rᵢ was characteristic of a deterministic misidentification, i.e. protein i is consistently mistaken with another specific protein j, and conversely a low rᵢ was indicative of a rather random misidentification. As shown in the right panel of FIG. 6A, proteins were often confused with several others, suggesting a relatively high degree of randomness in misclassification, while only 10% were consistently misidentified, that is with the same partner. The distributions for all other conditions are shown in FIGS. 6D and 6F.

### Example 3: Identification of Plasma Proteome and Cytokines Panels

The performance of this approach for clinically relevant applications, including whole human plasma proteome and a cytokine panel, was evaluated. In both studies, the CNN training was kept at the whole human proteome, rather than restricting it to the clinical subset. Next, nanopore translocation traces of the plasma/cytokines proteins were presented and the classification accuracy was evaluated as before. Interestingly for the high-spatial resolutions (20 nm and 30 nm) the correct ID of the 3852 plasma proteins was only slightly larger than the whole proteome accuracy at the different labelling efficiencies, reflecting the fact that there is a small set of proteins that are hard to be classified in both cases (FIG. 6A-B, right panels). However, at the lower resolutions, especially for the 100 nm case in which there was observed a significant drop in the ID accuracy for the whole proteome results, very high scores for the plasma proteome were still obtained. Even at the lowest labelling efficiency of 60% at 100 nm resolution the CNN classified correctly 93% of all plasma translocations (FIG. 6B). In addition, the fraction of proteins correctly identified with probability between 0.9-1.0 improved over that of the whole-proteome classification, reaching 96.8% for the case of 30 nm resolution and 80% labeling efficiency. Finally, close to 30% of misidentified proteins were consistently mistaken with another specific partner, suggesting that the accuracy of classification could be further significantly improved by relaxing the requirements of correct ID for selected proteins. These results indicate that single-molecule plasma proteome application, which holds great clinical value, does not require extremely stringent experimental resolutions or super-efficient labelling chemistries (FIG. 6G-I).

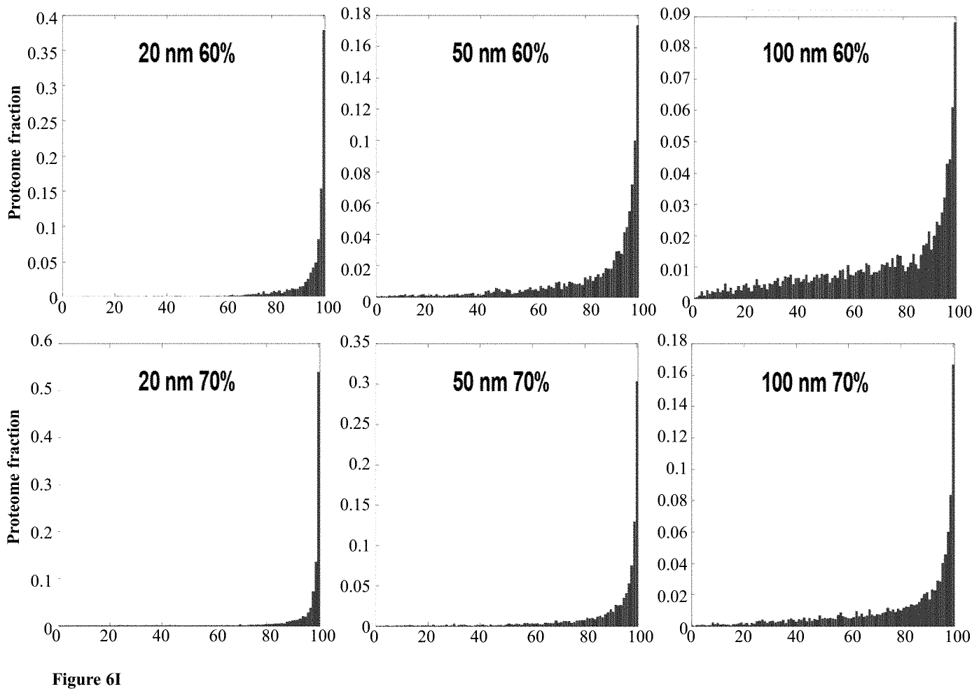

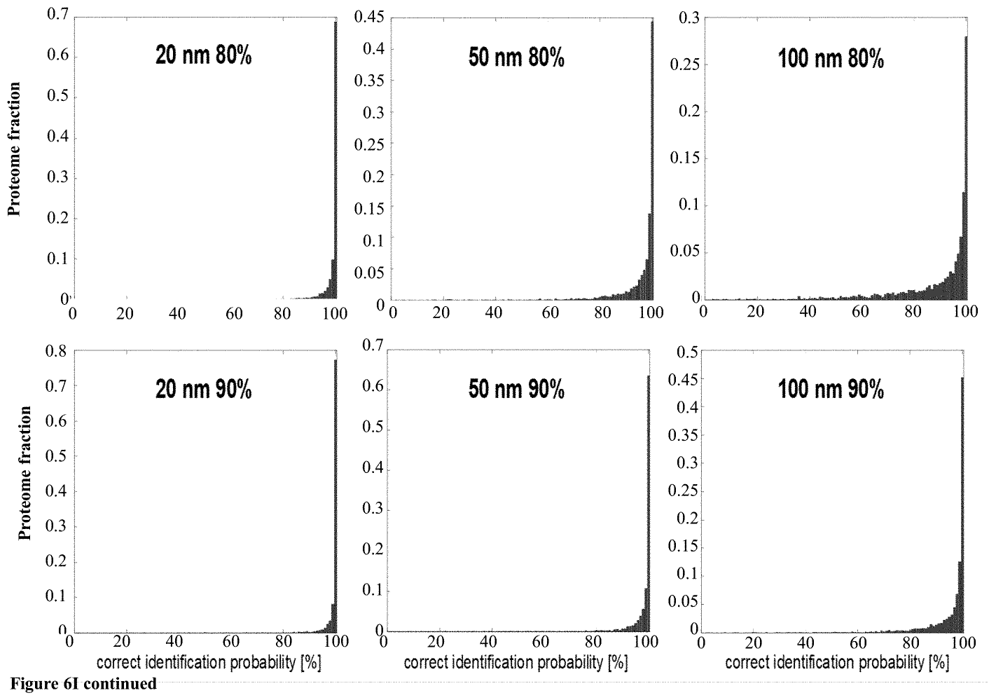

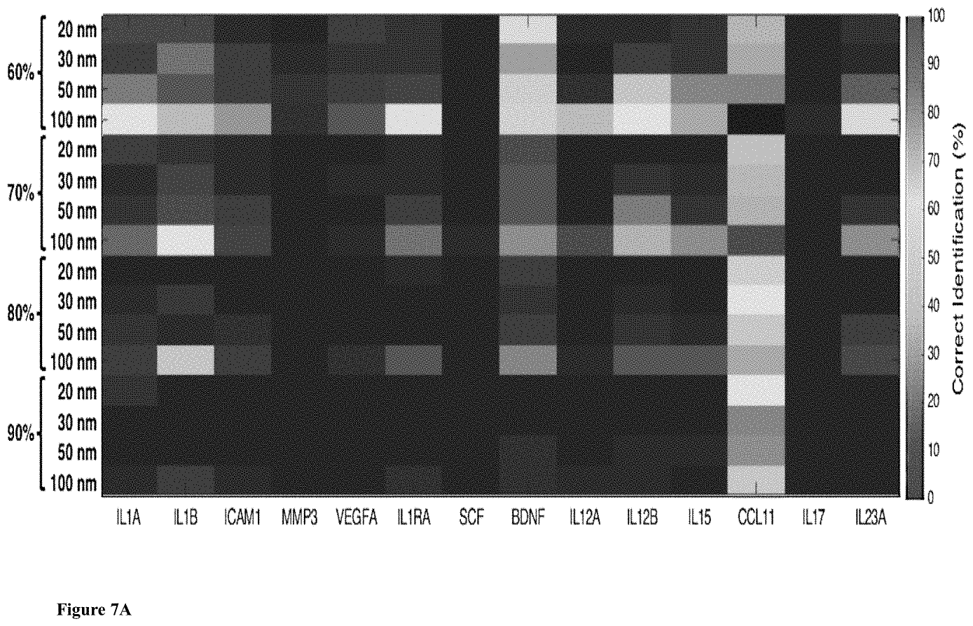

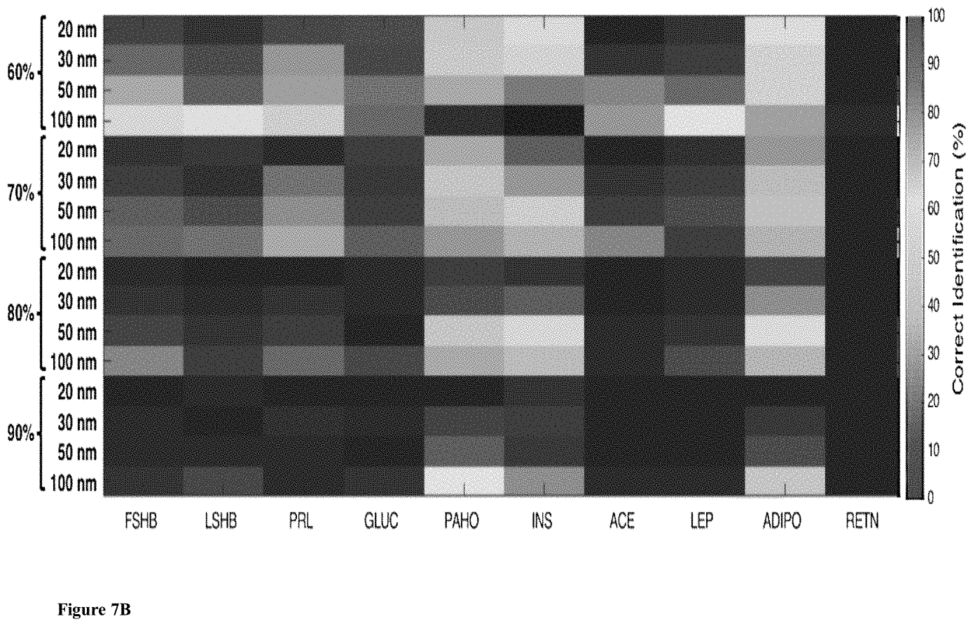

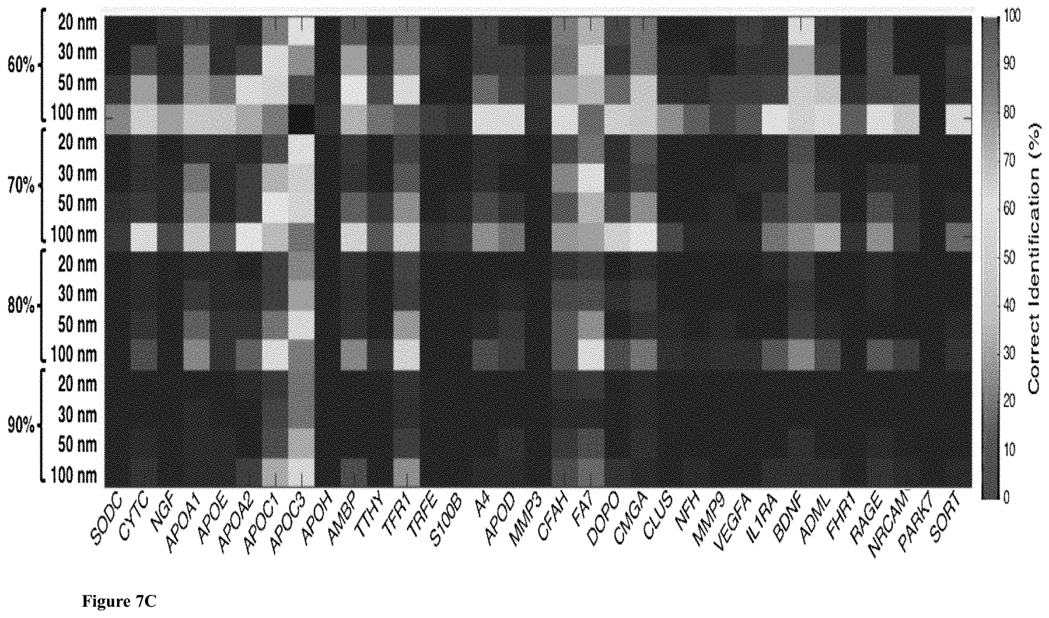

The cytokine panel (CytokineMAP) contains 16 proteins involved in inflammation, immune response and repair. The CNN classification was evaluated under 16 different experimental conditions (FIG. 6C). At the lowest labelling efficiency of 60% the ID accuracy drops between 43%-85%, and at the realistic 80% labelling correct ID was obtain in the range of 73%-97%. However, despite the functional similarity between the candidate cytokines, and the wide range of conditions tested, each was distinguishable from all other cytokines within the commercial test panel. This indicates that this approach has the potential to meet the requirements of a broad range of clinically relevant applications—that are less demanding than whole-proteome identification—with extremely high accuracies and yet very poor experimental conditions (FIG. 7A-C).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of identifying a peptide, comprising:
a. receiving a linear readout representative of at least a portion of a first amino acid and at least a portion of a second amino acid along said peptide; and
b. analyzing said linear readout with a machine learning model, wherein said machine learning model predicts the identity of said peptide;
thereby identifying a peptide.

2. The method of claim 1, wherein said portion is at least 60%.

3. (canceled)

4. The method of claim 3, wherein said machine learning model is trained on linear readouts of a set of peptides, wherein each linear readout represents at least a portion of said first amino acid and at least a portion of said second amino acid along a peptide from said set of peptides.

5. The method of claim 1, further comprising labeling at least a portion of said first amino acid with a first label and at least a portion of said second amino acid with a second label along said peptide and detecting said first and said second label linearly along said peptide to produce said readout.

6. (canceled)

7. The method of claim 5, wherein said detecting comprises passing said labeled peptide though a nanopore, wherein said first and second labels are uniquely detectable as each label passes through said nanopore.

8. The method of claim 7, wherein said label comprises a fluorophore and an optical sensor at said nanopore is configured to detect fluorescence at said nanopore, or said label is a bulky group and an electrical sensor at said nanopore is configured to detect electrical current and/or voltage at said nanopore.

9. (canceled)

10. The method of claim 7, wherein said nanopore contains a plasmonic nanostructure, wherein said plasmonic nanostructure is configures to localize electromagnetic excitation below a wavelength of light, to amplify localized fluorescence emission at said nanopore at a plurality of wavelengths or both.

11. (canceled)

12. The method of claim 7, wherein said nanopore has a resolution of at least 100 nm.

13. The method of claim 7, wherein said linear readout is a linear temporal trace of said peptide as it passes through said nanopore.

14. The method of claim 1, wherein said peptide is an undigested or unfragmented protein.

15. The method of claim 1, wherein said linear readout is further representative of a portion of at least a third amino acid along said peptide.

16. The method of claim 15, wherein said first, second and third amino acids are lysine, cysteine and methionine.

17. The method of claim 1, wherein said set of peptides is a set of peptides selected from:
a. a set of peptides with known sequences;
b. a set of peptides expected to be in a sample and wherein said peptide is from said sample;
c. proteins found in plasma and wherein said peptide is a peptide found in plasma; and
d. proteins found in a proteome and wherein said peptide is from said proteome.

18. The method of claim 1, wherein said linear readouts of a set of peptides comprise at least 50 linear readouts representative of each peptide from said set, are simulated linear readouts based on a known sequence for each peptide wherein at least a portion of said first amino acid and a portion of said second amino acid are represented in said simulated readout or both.

19. (canceled)

20. A method comprising:
at a training stage, training a machine learning model on a training set comprising:
(i) a plurality of linear readouts, each representing at least a portion of a first amino acid and at least a portion of a second amino acid along a peptide, and
(ii) labels identifying said peptide associated with each of said linear readouts; and
at an inference stage, applying said trained machine learning model to a target linear readout representing at least a portion of said first amino acid and at least a portion of said second amino acid along a target peptide, to identify said target peptide.

21. The method of claim 20, wherein said training set comprises linear readouts
a. of a set of peptides expected to be in a sample and wherein said target peptide is from said sample;
b. for at least 15 peptides and at least 50 readouts for each peptide;
c. which are simulated linear readouts generated by selecting a known sequence of a peptide and generating a linear representation of at least a portion of said first amino acids and at least a portion of said second amino acids along said peptide; or
d. a combination thereof.

22. The method of claim 21, wherein said training set comprises linear readouts of all proteins found in plasma, or all proteins found in a proteome.

23. (canceled)

24. (canceled)

25. The method of claim 20, wherein said liner readouts further represent at least a portion of a third amino acid along said peptide.

26. The method of claim 20, wherein said linear readouts comprise a linear temporal trace of a labeled peptide as it passes through a nanopore, wherein said peptide is labeled at least at a portion of said first amino acid and at least at a portion of said second amino acid along said peptide.

27. A system comprising:
at least one hardware processor; and
a non-transitory computer-readable storage medium having stored thereon program instructions, the program instructions executable by the at least one hardware processor to:
perform the method of claim 20.

28. (canceled)

29. (canceled)

30. (canceled)

31. (canceled)

32. (canceled)

33. (canceled)
